SEQUENCE LISTING (20 sequences)

SEQ ID NO: 1 - DNA, 1384 bp, Plasmodium berghei
ctgatgggttacattttgtaagaaataagaaaatatgtttagtattttaatatattactgtagaatttattttataacaataagacatataaaatataattataaaaaatattatatatataattatttaatataatataatattttaaatataaactaatataataattaatatactaattaaataataatatttaatataaataatttatatattaaaattaataattaaataataattaataatataatattttaaatataaactaatataataattaatatattaattaaataataatatttaatataaataatttatatattaaaattaataattaaataataattaataatataatattttaaatataaactaatataataattaatatattaattaaataataatatatagcttatatatcaattaataattattattatcgtttaattaattaaatataaaatataatattaaatataaaataatatattaattaatattaatattttaatataaattaataaataatatataataattttcatatttaaaacatgaattatgaataatttcttattgatattaatatattaaaaataaaataataattagtatactaaataatatttattttttaaataatgtaaatattatattttaacattaatatttttaaataattaattatttattttaattattataaataataaaataatatattaaatttattatattgtgtgtgtaatttatattaatttaatatatattaattatatactatttaaaaataataaaatataaattacatttaattataaaatttaaaattattttaaataattaacgataattattatattaaatattttaattatattacatttaataaataaattaatcttattttaacttaattatttatattaattaatttatgttttaaaatattatttaattttttaatattaatattttattattattttaaaactttaatatttattttaataaaaaaatatatattaataaaaatatatatattaataaattgttaattttccatttattattttaataataattataatatttattatataatatatttaaatatttattaattacaacaataattttattatttattttaatttattattttaaatatttaaatgttattcactacataaatattatttatattaattttatagtatttaaaaatgtattattaacatttaattgaccatatctattatgttttgataattgttattttttaattaaaattatatgtgagtttttacatatatttataattaatatatcaaaattattttaaggtatttgtatcataatcattttgtttgtctt

SEQ ID NO: 2 - DNA, 3016 bp, Plasmodium falciparum
ataacttttaatacaaagaatcctattaattactgtatgttaaaatggttgaatatatattcatataataaaaaaatgaaaaatatgttaattataaaaaattatcaaaattagacagttatatgaaatatttacaaatgtattttttaagaattcaataattatttaaatcactattacatacaggtctattttttatatatatattttcttaaataataagcatataaaaaaaaatgcttctaattttttattatataataaaaataatatacgaatatgaaaatattattaaacaataatagacattatataataaataaaaaaatacatataaaataaataaaggatatttaaaaacaaaaaaagactgataaatatgtttttttttttaaataattgaacttaatataaaaatatatatataataactaaaaaaatattattaatataaaaatttaacataaaaatatattaaattaataatattaaaataaattaattttagtttattaaaaatataattattaaattatatataaaatatattaaaaaatataaaattaaattaattccatataataataaatttaattattttaaataaatacatattaataaaaatttatataataatttatgtataattaaattaaatattataaacacacatatatattatttttacatttaaataataaaattaagaaataatataatttatatttaataaaataattattaaattaaataaatattttaaatatataaataaaattaatttaaaaataaatatataatatattaaaataaataattatatatataattaatatttaaattgttaataataaattaaataaataaataataataaacaattaattaataataaataaataaataataataaataaataaataataataaataaataataatattaattaaataaataataattaattaaatacataaataataataaataattaaataataataattaaataaataattaattaaataataatattaattaattaaataaataataattaattaaatacataaataataataattaattaaataaataataaatatttaattaaataataaatatttaattaaataataaataattaattaaataaataataaataattaattaataataattattaattaattaaataataaatatttaatatattttatatataattgtatataattaatatttaaattgttaataataaataattaattaattaattatattaaataaaaaataataaattatattttaaattaaattaataatatatttgtaataaaataatatatattgaataaataatactaattattatctaatgtttattattttaattatattaaataatacaatatctaaattttaacttaataataattaatttaaattaataaaatattaaataatatatatataattatgtgtaaataattaaaaataatatatataataattaatttaactttttatttaataaattaaaatattattaaattataaattattattatattatataaataaatatatatatatatatatatttattattatttaaaaataataatttaataataataattaaattaaaaaatatatatttgttgtacaatattaattattaaatatttataattagttaattaattaatttaataatatattaaatatatattatttattttataattaaataaataataaattaaattaattatatattatttatttatatttaattaaatatttattaattaaaataaataatattaaataaatatatatatatatataattattattaagataaattatattaattaatatataataaaataattattttaataaattaaataaaattaaacaatttaattaaataattatatttttaatataatttataatatattaataaatttattattattttaaataatattaattaatatatttcatttattattttataatatacaatataatattaagtctaaataattaatatattaataattaaataatatttaatttaatttaatgaaacaaaatatatatattttcttaattatttaataaatatatattaaaatatataataattatttaaatttaatatattaatattttttattaaattaaataataaaataatgaaatttaattaataattatatataatattaaaattttattaatttaaaaattaaatattctatataatttattaaataaataataaaatttaaaaaataaataaatattaaaataatataatatttaaaattaaattaataatatatattatttaattttattttacttatttatataacataattttatataccatatatttaattaatttatatatttaaataattaaatatataatatatttattcgattttatttatttatataaatattttaattaaataattaattattattttaaaataaataattaaagctataaatattttttaatttaatatattattttaatatgtgtaacattttattaaaataaaataattattttaaaaaaataataatttaatatattaaaattaaagttattatattttattatttattatatgaataatataaaaattaaataattaattttatatatttattaaattaatattaaattaaattaaattaagataataaatatatttgttctttattttattattatcttttaaaaattatattatacattttataataaataattgttaatgtaatacaataatttatttttaaatagtatgatttatataaaatgaaaaggaaaatattaatatattcactcatctaaaaacttttcaataaaaaatttatattttattaaaacattaaatgaaaaattccaattatttacaaatgaattatatttattcaaatgttaaaaagacattaatataaaaacaaaaaaaat

SEQ ID NO: 3 - DNA, 2737 bp, Plasmodium falciparum
tttcttaaaaaacgatgatataataaataatttaatatatattaattcatttatatatataataatatatttcacattaattttaatagtttatatataattatataatttctttattaataattaattatagtacacatttatattaataaatataattaaagaatattttatcttttgtattatatatatatatattaaatggaataaattaaataaataaaataataaaaataatatattaaaatatataaattaacaaaataataatataattaaataaataaaatattatattaaataaataaaataaaataaaatattaaaaaatataaattaataatataattaattaaataaaatattaaaatatataaattaataatataattaataaataaaatattatattaaataaattaaataaaatattaaaatatataaattaataatataattaataaataaaatattatatatattaaataaattaaataaaatattaaaatatataaattaataatataattaataaataaaatattatattaaataaattaaataaaatattaaaatatataaattaataatatatataattaaataaataaactattatattaaataaattaataatataattaataaataaaatattatattaaataaattaaataaaatattaaaatatataaattaataatatatataattaaataaataaaatattatattaaataaattaataatataattaataaataaaatattatattaaataaattaaataaaatattaaaatatataaattaataatataattaataaataaaatattatatatattaaataaattaaataaaatgttaaaatatataaattaataatataattaaaatattatattaaatgaattaaatgaaatattatattaaataattaaaatattattatattaaataaattaaatattatattaaataaataaaataatatattaaaatatataaattaataaataaaataataatataattaaataattaaataaaataataatatattaaataaataattaaataaaataataatatattaaacaaattaataaataaaataaaatattatattaatttatatgtttaatatattgtctttattatttaatataatattttaaataaataaaaatatattaatataataatttaataaataaaacaatttatattatattaaataattaaatatataaaattaatataataatttaataaattaattaaattatttaatatataaaataaatatatacgctaataaataattaaatatataaaaatataataataaataaaattaaaattattaaattaattaaataatataataaattaattatttaatataattaatatataataattaatttatttattttagtaacattataataaaataatatatatatataataaattaatatatttattaaaataaaaacaaatatatatttattatatttttaatatataaatattttcattttatttattatatttttattaattatatttttaaataataaattacacatttaattaattattatttatttaattataactttataatatttaaatataatattaattattttatattttttaaatatatatataaataattataataatatattttaattaattaaataatttatatttaaaatgtgatatatatatatatatatatatttatatatgtaatataattaaatattaaatttttattttatatataatatatatttttatataaattatattaataattattaaaatataataatatattaaattaactttaaaataatatttatttattaatgtattatatcattattatttatatccattttatttaattaatttattattttaaaataaatatattaattaaatatattataaataatatattattttttatttataataataaaattattatatttaattaatatacttatattaaaactaaattaattaattttaatataaattaatatttaatatatttattaattttctttttaattattaaatttatatttaaatattatatatttattaaataaaattaatatatttttacttatttaaatattaattttatatttaattaataatatatttattaaataaaattaaattattttttcatatattaaatttatattaattaataatatttataatgaaattattatttataattaattaatttatattatgtatttattattattaaaattatttttttgtgtgtataatatttaattaagattttatatattgatgtttatttttataaattaaataaaataattttattatattaatttaattaattttttttaatataattttaataaattattttatatataataattaattaaattattttaaatataaatattaaaagtttatatttaaaactattaataatttataatttttctttatttaattttattttttctttattttatttaaataaattttattaatatatatttattgttaagtatttaattataatatttatattaacacattggaaatttatgcttttattttataatgtaatttatttttaataattatatattatattattattttttatgcggaaattattatacattaaataacataattgattcttttat

SEQ ID NO: 4 - DNA, 2746 bp, Plasmodium falciparum
aacaaataggattaaaatgttacctttttatatattttctgaattaaaataatttctttcccaatttctaattatataaaaatatatatatgatattatcatattatataattatatttatttaaataataataatatttattgaaaataaatatcttaaggaaaataacatatatttatattagaagtaaaagaaaattatttttatttaatataaaaaaagttatatataaattaatatgcataaatatataatatattaattaaataataattaaattaattataattatgtataaaataatttatatattaattaatttttatttatatatttattaatttaattatacttaaataaaattatataattatttattttatatttattattaaattaattaatataaacaatataataatttattataggtaattaattaaatatattaaatatattaaaaataataatatatttaatataaaacaaaaattaatatttaataattaattatataatatatatatatataatattatttattattattttaatataaaaatatattataatatattaattattacactgttattttatatattaatattataatttatttaatatataatatatatatttaatttaataaaaaagaaaatatatatgtttattaatttaataaataaattatatatatattacatatataatttaacttaaaataaattaataataaataatttcatttattaccatttaatttaataataataaaaatatattttaaattaaaattaattataaattaattataaatatatatattttatataatttaatatttaaaatataattattaatttataataaaaataataaaatataaatatattaatatattattttaattaatctatttgttatgtataataataaaactattatatttaaatatataaaatgtgtatataaataaataattctttttaattttatttttataattaaattattttatttaattattattatatatatataattatttattttttaaatgttaaataaataataaaataataatataaattaattaattaaataaaaataataattataattataatatatttacatataaaatatatatattatatatatttaatttattattttttatttaaatgaataataaataattaattatattaatttaatttatattaaatattatttatatatatttaaattaaattaatatatttatttttaataaacttaattaattaattattatttataaaaaaaatatattaaattatgatatatatataatattaatatatatttaattaattaattaaaattaaaaatatattaaattattttttaattaaattattatatattatttatgagtctatttattttattttttataattaattaattaaaattatatatttaataatatattttgtttctttgtattaatatattattattttatttatattattaaattaaattatattaattaataaaaattataatattatttcatttattttaatatatatttatataataataattatttccaatataaataaataatgtatttattaaagttaatataattattaatttatttttataaaataaatattgctttaattcattaattataatatttattatattatttttaaaataattatgatataatattatatatattaataatttaattagtatataaaaatttaaacatatacattataatttacacataattatttattttattttaattaatttttacttattttaatttaattattttattttatattatttatttattattttaattaatttatttaatatattaatttatttttattttatttatttatttttattttatttatttatttatttatttttattttatttatttaattattttaattaatttatttaatatattaatttatttttattttatttatttatttaatatattatttatttttattttatttatttattatttaaatttatttatttaatatattaatttattttattttatttatttaattattttaattaatttatttaatatattaatttatttttatattatttatttattatttaaatttatttattttatattttaattaaattatttattttatattatttaattattattttaatttatttattttatattatttgtttattattttagtttatttattattttaattaaattatttattttatattatttatatattattttttaatttaatttattatattaaatttatttaatatttaatattttaattttatttatttaattaattaattatttatttattttaatataattatttttaataaatatttattatatattaatttaaaaatatatttttaaatttatttttatttaatcttatattttatatttacacacatttatatttataaaaaaaatatattcttttttaatatatatattaaataattaatactaaaaattaaaatatatataatgtatattatttaacataatatatattttttttttttttatttaatacaatttaatttatatattaaataatataatatttattataaataatatataaaatttagcaactttgaattgaaataatgatatatgaatttttaataaatataattttataattttac

SEQ ID NO: 5 - DNA, 2827 bp, Plasmodium falciparum
caaataaaatatatatatagtataaagaaatattaagaactatattatttatatatacatttttttatttaattattaatatataaataatatttaatatacaaatataaatcatgcttttaaaataaatatataatttttattaatatataaatataagtattatttatatgttataatacattttattttaattctttctaatttaatattgataacatatttttataattaatttacacatttattaaaaaaacattatattaataatattaatttatattttaaaataaaatataaatatttaataaaataataaaaaaaaatatatgtaatagttatatatataatattaaattaatataaattaatataatataataaataaataataatatatatattaaataaataaaataaacaaaataaattaaattattttaaattaattaaataaataaaatatattatttattaaaataaataaattaatatatattatttattaaaataaaaataaattaatatatatatttattaaaataaaaataaattaatatatattatttattaaaataaaaataaattaatatatattatttattaaaataaaaataaattaatatatattatttattaaaataaaaataaattaatatatattatttattaaaataaaaataaattaatatatattatttattaaaataaaaataaattaatatatattatttattaaaataaaaataaattaataattaaaataaataaattaatatatattattaattaatataaataataaataaataattaaaataaataaattaatatatattattaattaaaataaataataaataaattaataattcaaataaatatattatataatatatattaattaaattaataatttaaataaataaatgttttttattaattaaattttataataaataaattaataattcaaattaatataatatatattaattaaatgaattaaataagcagttataataaataaatatattaataataaaataaataatttattattttattccacttaatatatatattaaaataataattaaattaaataatattttaaaatataattaataattaaatttaattttatttataatattataataacattttaattcatatataattaattcttaataataattataataaataatttaaattaaaataaattaaatgtttacttataaataaattaattaattcattttaattattaatttaattaatataataattatcatatttatttattttattaaattaatttaatatataattaaataattaatttataataatatatatagtatttttaattatttattttatgaaacatatttatattttttaattattgaatacaatattaaataaattaaataatatatatatattacacacacgtattattaaatactgttttaatttaatttaattaaaattaattaattattattatatataattattataatataatatattattaattttattttattttaatatatataattatatattatattataatataatatattttattatttattaatatatttaatttaattattttatattttttaactttaattaattaatattaatattaaaattaaataattatttaataatataattttaattaaatatatttaatttaattattatatacttattaagtattttattttaattaatatttatttaatttaattatttatatttaaagtattttattttaattaatatttatttaatttaatttaatttaattattattatatataattattttattatatttattttattttaattaattaaataatatatttaaatttaattatataatatttattacatttattttattttaattaaataatatatttaaatttaattatataatatttattatattttaattaatttaataatataattaaatttaattataattatatatttattatatttattttaattaatttaataatataattaaatttaattatataacatttattatattttaattaatttataatatttattatattttaattaatttataatatatataaatttaattaattatataatatttattttattttaattaatttaataatataattaaatctaattatataataattaatttttatttatatatatattatatagttaattattaatattatatttatttaaaattattattattatatgtatttaattattttatgaaatttattttaattatattttataatatttaatattttatatttattatatatatattatacacttaattattaatattatatttaatttaatttattatatgtatatttaattattttatgaaatttattttaattgtattttataatatttaattatattatataaaattaaatttattattatatttattttaataaatgattattttattgatgtgtatttattatttatattataatattttgaattaaatataattttaattaataaattgctaaattaatttattatatgttatattgtttatattaagttaatatattatttattatacaaaatatatatttaatttatataattattcttattatacaattacatatataaatattttaaattgaaaaaaaaattcattttatttatacagtatattaaaagaattaataatataatatttttaaaaaaatttaatgggttagataaatattaacatatatatattaattat

SEQ ID NO: 6 - DNA, 2890 bp, Plasmodium falciparum
atattattttgttttagtttatattatacacttcatataaacatataatatatatgttagtaataatataatattttgattaattgcaaaattatatatatatatatgaaatattctttgggttaataattaaatataagttatattattattatttttcttaataaagaaataatattaataaattgtgtgataagaataatttacaattttcttttattaatgataattatataaaataaataaatatataattatatataattaaaaataagacattaaattaatttattaaaatatccatatttatttaaataaattaaaataatattataattatataaatactatttatttaaaaaaaataaaataatataatatataaattaaataaaattaattaaaataaaataataattatattaattaaataaattaattaaattaaaataaaataatagttatataatatataaattaattaaaataaaaataataatcatataatatataaattaattaaaataaaataataattatataatatatgaattaattaaaataataataataatcatataatatataaattaattaaaataaaataataattatataatatataaattaattaaaataaaataataattatataatatataaattaattaaaataaaataataattatataatatataaattaaattaaaataatttttatattataataatataatgtatttaaataaaataatatttatattaataatataatacatttaaataaaatgttatttataaaatatataaattaaattaaattaaaatttatattaataatataatattttaaataaaataaattaatataatatattaaatatattaattaattagaataaaataatataaaatatataaataaattacataatataatatattaaatatataacttaattaaataagataatataaaataaattatatgtgtgaattaattaaaataaaataattaatataaaatataaaaataaaataaattataaatgttaattaattaaaataaaatataaataaattaaataatattatatatatatgcattaaaattaaataatataaaaatatataaacaaattaaataatgtaacataatatattaatatataaattaattaaaataatataatagaaatatataaattaaataaattaaataaatataatttaaatattatataataaaataaataaaattaaaataaaatatataatattattaatatatattaaataatgttttaataatatatttataattatataataaatttaaataataaaatatatatatttatatattaaatataattaatttaaataatataataaattatttaaataaatattaaataaaataaataattaattatatttaatttataatataattattatattatttaaaataataaatattaattaatataaataattaaaacaataaataaattaattaaataaatatattatattaatataataattaaattaaaatgatataaaaaaataaataaatatcaataaattatatatttaaattataatattataatataattaattcgttaattttattatttattaatttatttgaaaataatatataattaatttatttaatataatatatatatatatattttaaataattaaattaaataataagaaaaatatttatttaattttatatatgtgtataaaaaaaatataattaattaattattaaaaattaaataagacatataaattaataaatatataatattaattaaattaatttatataatagtttataataataatttaatttattaattatttgttatataaatatataatatatttaaaaattaattaaaaaaaaataaatatattaaacatttaattataatttaatattattaaataaattataatatattttattaattattacacaaataaattaattttatttaaatattaaaaaattatatataatatattaattattttatttaatttataaaataaatattttttaattatattatagatattattatttaaattattttaatcatttaattattttatttaatattataataaattaattataattaattaattttatttattaaaaatattatagattgtatattaattaatttaatttaattaattaattattaatttaatattgtaatttataaattaattttaattaattattattaattttatttaatattataatttataaattaattttaattaattaatttaattaaataattaataagaaattatttaatattatatattattaattaaaataatttaagtattttaaattttaataaatattttaatattaaaatgaaattataattaattttattatatattaatatttatttttatatattatattatattattaaattattttatttaaaataaataattaaatatattttattatttaataatattaatttaatttattaaattaaaaatataaatatatattttttttattttttataacaatattattaaattaaataattattaaataataaatgaaaatttatataattattaagagtaaaattaaaataaaaataaattttataagaaatataaaattaacattttttatagataatataatatatatatattattattttttttttttgatacaaaactttatacacaacttataaataaatatataataaaataggatttaatataaatattaatcataattaatttttatccaaattaatatacatgtattatgaaattcttaatgaataaatcaataaggaa

SEQ ID NO: 7 - DNA, 2845 bp, Plasmodium falciparum
attctgaaataaatacttaaaaatatagattagtgttattgtagatatttgaatataaagctacacaaatacatagaatataccattatataaggaaatgcattaaaataattttataaatattatatatttattttaatttataagaaataaatatgttgtaatattatatatataataatatataagaatcatattacacattttataaataattaaaattaaatgtacttttttatatatttttaaaacagtaagtaaaataatataaaaattttaaatattaattaataaaatagaaaaatattatataataattaaattaaattaaataaaattaaatataatactaatataatttaattttaatattaaattatttaattataattaaattaaatataataaaaataataaataaagtaataaataattatagtttaattaaataaaattagattaaatataatattaaattatttaatttatataattaaattaaatataataaaaataataaataaagtaacaaataattatattcaattaaataaaattaaattaaatataaaattaaattatttaattataattaaattaaatataataaatataacaaataaataaataaagtaataaataattatattcaattaaataaaattaaatataatattaatataatttaattttaatattaaattaattataattaaattatatataataataaattaaattaaataatatataattaaatataacaaattaaataataaatatttttattttaaattaaataaacttaatatcaaataataaaataataaatatttaaaatataaaattataaattaaaataataaaataaataattaaaataatatataaataaattaaaataatattaaaatataataagtataaataattatgattaaattaataatattaataaataaaattaacaatataattttaatcaaaattaaatataataaataattaattaaatataaaataaaataaaaatagtaaatataattaatataattatatataatttaaataaataattaaattaataattaaatatgatataataaaattaattaaaaattaatataataataaaaaatataaaataagaaataattaattaattatataaattaatattaaataatattaatcatatattatagatttaatatatattaatttaaaataataatattattatttattattaatatattaattaattaaattattttaaataaataataattaaataaaaatataatatatatttaatttaaatattatataaattaataaataatatataattattaatttattataataaaatatattaatatttaaattaataaaataaataataatatatatttttttgttaaattataatacatatattatcaatatataataatttattttattaattaatttatttatattaaatatattaatttaatgtgtttgtctttaaataatataaattattatttaattttaattattttatttatttgttataatgattaaaatgaattaaattatatttaaataatttattttatatattaattaaatatttaattatatttattataattaatttaattatatttaaataatttattttatatattaattaaataatttaattatatttattataattaattaaagaattatttttatatttatttaattaaatgaatttattataaatgtataattaactatttatttttaattaatttaattttatatttatattatataatatatatattattaatttaatataattaatatatttaataattccttttttttatttattaatattattgtcttataatatataattaatttaatttattatttaatgttattataatatattatttattaaatataataaatttaaaataaataattaattaattaatatttaatataattaaatattatatatatataattaaattaattttattatattaattaaataaatataaataaattttatttaatttaactattttaatattattaaataataaatatattaattttattaaatatttaatttattattttataaaaattataatttattatttaatttaattatatatatatatatataaatatatatatattttaatttatttattataattagttaaattatataatttattttttaaattaattatttatataattaatgaatttatttattatttatttaatatatattttttaataaattaataattaaatatatattttaaaattaaattttatttattaaataaatattcttttttaattgtttcatattatcaatttaattaataattaaaatatttaatatattaattatatatattatatttttaaaaattaaatattaaatttattattttacttaaaaataaatattaattaaatatttatttctaattattttttaattatatataatatatttttataatattatattaaaacttaatatataaaaaatattttatctgataataaattaatatatattaattaatacgcatatttagatattttaatttcatataaaattaaaatagacttttttatttaaaaaaattactttgcattttttcttttattaagagaatttattattattttttttttatattttatttctattattaatttaatataagtgtataagacattttaatatttttatcataagttataatacaaatatatgatgaattattataatttttagatgtgtaacattgtttatatg

SEQ ID NO: 8 - DNA, 3259 bp, Plasmodium falciparum
tattcaataaattatatttacacatattctaattttgtttttttccctgtaaaatatatatgtgattctgtatataatatatcatagaatatatttattattaaataataatttagattttacttcttaaacattatatattttataatacataataaattatatataaaacaatgttaacattttaaaataataaataaattatattaaatttaaataaaatattttaatatatataatatatattataataaaatatatatattatataaataaataattctgtttattttaattatatttaattaattttattatttaatataataacatttaaagatatatataataatttaataaaaatattataaaattttataataaaacaaataaatattaaatttattaattatttgtattatataaaatgtatataaacataaatatttaattatttttttttatatattaatttaaatattttatatttattaataatatttaaaaattaaattaattaaatatatttcttattatatcattaatatatatatgataatacttattatatttaaaataaaatatttatttttaatttattatcatttaatatattatatatatttttttttaatttaattaaattaatatatattaaaaataattaattataatattattttttttatattaaattaataaatattaattatattttaattaaattaatatatatatatatatatgtgtatttattattttaattatatatttattaaataaattataattattttattattaattaaaaaataaataattttaatatattattttatttaattataagaataaaccattaaaaaatatatattattatatttaattaaattaataattatttattattaaatataagctttattttattttaaaaattataaaattaattataataaataatattaatttatattttaatatttatttatattaaattgtgtttaattaattataatatattatttatttatttatttaattaaatttaattaattaattaatataataataattaataaattaaaaataaatattttattttaaaataataattgttattaatatttattaataaaataaattatatatttaatttaatataatttatttattataatatattatttattttatttaaaatatatgtatttatttatttataatatattatttatttaatttaattaattaaattaatataatactaataataaattaaaaataaatgttttattttaaaataataattattattaatgtgtattaataaaataaattatatatttaatttaatatttatattattattttatacatttaatttatttattaatttaattgttatatattttaatatattataataattaattaatttaattattgttttaatatattattatataaataatattattttatttattggtattaatataattgtttaattataattttctaaatattatttattaattattaatattttatattaattatatttttttaatattaatttaatcatatgctatatatttaaatatatttaataaaataattctttatattaaatatattattttattttaaattaaattaattatttaattatttatatattttaaaaaatatttattaaattatttacctaatatatatattttttaattttatattaattatataatattatttaaaatgatttaataaggaattaatgatatatataattatttaattattattttaaattatttaattaatatatatatatttaattaataaatatatttaattattatttttaattatttaattaataaatatatttaaatattattttaaattatttaattaatatatatatttatttaattaataaatatatttaattattatttttaattatttaattaatatatatatttatttaattaataaatatatttaattattatttttaattatttaattaatatatatatatatatttaattattatttttaattatttaattaatatatatatatataattattatttttaattatttaattaatatatatatatatttaattgttatttttaattatttaattaataaatatatttaaatattattttaaattatttaattaatatatatatatatatatatatatttaattgttatttttaattatttaatataaatatatttaaatattattttaaattatttaattaatatatatatatatatttattattttaaattatttaattaataaatatatttaaatattattttaaattatttaattaatatatatatatatttattattttaaattatttaattaatatatatatatatttattattttaaattatttaattaatatatatatttaaatattatattaaattatttaattaatatgtgtatttaattcttattttaaattaattaatatatatatttatttattcaattgttttaaattatttaataaataataatattgttttttttaatgaatttaattttattatatattattatattatattatatatttttttttaataattaattttatttatttatgtgtatatttttttttatattccattttacattttttataattatattatttataaatgtgtaaactttatataaaaatttatatatattaataatatatttagtagtaataattatttttatatttcatcttaaaaaattcatttttaactaaatgtatttaaaatatttatataagagataaagatatgattcaaatgtaaaaatatattattgaactttataagattttataatatgttttaaacaaaaattgtacaatataatcttcctattacagtaataaaaataatatatatatttacaaagaaatatgtctatatgaaaaagatatattaatatataatatataatagtatgaaaactgtgaaatatttatatatatatttgttggagttttaaactttaaataatatttttttaaatatagaatacataaataaataaataaatagtaaatacataaataaaatttatatttaataagaaaactcaaaaaaaaaaaaaaaaaaaaaatatattatatatataatatatattcatataattatcttaatttccatgttatgttcactaaagttgtgttctattttgtttgatcatca

SEQ ID NO: 9 - DNA, 2755 bp, Plasmodium falciparum
aatctttcatacaccataatatattaaaaacattataaattatattttccttcatttaccaatatattttattctaaatataataataaaatataataacaatattacaattattaataaatattattttcattaataaattattaataaataaatattaattatatatatatatatataatattatttttattaaataaaatgtataaatatttatttttgctatttatttaaattatataaattaaaatatgatttaaaaacaatttaatttattattttataaaaaaatattatttaatatatataatttaaataaataaaaaaatataaattaaattaattaataatttataattactattaaaaaaataataatatattattttaaatttattttaatttaaaagtaatataattaataaatatcttattattattatatttaataattaactataaatatgataataataaaataaatacattattttaaaatataatatataacaattaaattattaatattatttattttaatatataataatattatatattataattaataatatttaattttaattaatttataataatatataaatattttatttttaataaattaattttaattaatttcgaatttattaatttaattatataaattaatataaatattattttaatattaataatttaattaaataatatattaaattcatcattttattataaatacataaattataattatataattaatttattattttattaaatatattaatattataatattaatttaaataatatataattatattaattatttttatattatatataattaattcataaataaatatattattttattattatatatttgaatttaattaatttataataattattaaatattataaatattaaataataaataatatattattttatttattatatgtttgaatttaattaaattattattattatatattattaatattaatttatttaaataatatataattgtattaattttttttaaataatatatataattaaataaatatatataaattattttatttattatatatttaaattaaattaatttattattttatattaaataattatattaattttcaatttatatatttaatttaaaatatataattaataattgttaatttaattaaaaataaaaataattaatttaaatattatattaaaatatattatttttatttttttaattatttattaaatactattatattattattttatattaaataattaattaatttaatatattatttaataaatataataaatatattattatatatataacacacataattatatacttaagatatatttaaataataataattattatttatttttattatatatatataatattttattattaattatttaatttatataataaatatatattttaattaataatataaaataattaattaacaaatatatatttatttataaataaataataattctttataattaattaataattatattattttaattaatattttaattaatttttgtttgtatttatttaattaaattaatatattatgtgtatttatttaattaatttaatttattatgtgtacttatttaattaaattaatttatatttttagtattaatattatatatatataattaattatttattaaattaataatatatttaaataaaaagttaaaataatatataaataataaaattattaattaatttaattaaataatttaattaaataatatatttaaataaaaaattaaattaatatataaataataaaattaattaatttatttaattaaaattatatgtttaatttaataaattaattaattttattatttatatattattaattatttatttaattaattatgtataaattatttattcatttatttaaatatattaatttattatttcgttaattatatattaataaatatttatgaaaaataattatttaattgttagaatgatattatatatttaattaatttattatatatattaaattaattatatttatattgtattatatttatttatttatttaatttatttttatttttattttattttaattaattaattatatttatattgtattatatttatttattttatttttattttaattaattaattaattatatttatattttaattaattaattaattatattaatattttatttaattaattaattatatttatattttatttaattaatttatttttcttcttattcttattttatttaattaattaattaattatttttatattttatttatttaattaattatttatttttatattttatttaatttaatataataattaattctttatatttaattttttaataatagacatatattaatttaacatttttatgctatattatattattattatttaaaattatatattttttttattaaacattcctttatttatttatttatttatttattattatttctatactttatttttattttatttacatttttgtgaattattatttatataagaaaatatgactttaatttatataattaattttttgtttaattattgtatattttatataatatattaagtgtaatattagttattatataaaattgtatatttaaaaaaaataaatataatgtacttccaaa

SEQ ID NO: 10 - DNA, 2854 bp, Plasmodium falciparum
caatgtacaattataaataaataatgattctattctataatatttataaatttataatgtctatatatatatgaacactatttaaaaaatatataatataaatatatataatatattaaaaaaatatattaatttcttattataatttccatttaaaagtattactttaatataaagatatgagatattaataatacatagtaatgtgtgaaagatattttataaataataatttaaattaataagaatttataataaattaaataattaataacgataactatgtatataatatttaataaataaatataaattaaaaaaaaaaatatatataaataataatataataattaaatataaataaaattaaataaaataaattttaattaattaaaaaatatatatatatatacaaaattatacaaattaatttacaataattaaattattaataatattttaatttaattatttattaattattttaaaaaaatatatacaaaacaataaaatatataatataaaaatgttgattaaaaaaataataaattaattatatatatataataaataaatagctaaaattaaattacaaataaagtaattaaatataatattaaataaaaataataaattaaataataaataaataattaattatatatgaataattaaaaattaatatattaaataatttataattaaattaaatattaataaattaattaattgaaataaaaaattaataaataaattataattattaaataattaataaatataatattaattaaataataagataataataacattttaataatttaaatacataaaatatataaaataaatattatatataaatatataaaaataaataaataaaaaaataaataaataaataaaacattttatattattttaaaaaaattaaggaatattattaaataatatatattaatagttaataaaaatttaataaatatattattattatatatataattaatttattaaataaacaaataaataattattttaattctaatatttaataaaatttattatatatttaattatattcattataatttaataatataaattaaattatttaattatatatatataaatataaatatattaatttattaaatatattatggatgtgtaaaattaatatattattttaattaatgaataataaataaatattaatatatacattaatatatttttttattaattaataaatatttattttaattataataaaaaataatatatatatatataattaaataaataaaaataatatatttatttaattttattattaattttaattatatttaatttttaaattaaataataataattatatatattaatttatattttttaatattaattatataatatataattatatattgtctttttattattttgtattatatataattattaataattcaaaataaatattaaataatatttaattaaattaaattttataatatacataaactaagtattattaaatagtattataatatttaatttagttaatactaaaattaatatactttttaaattaaatatatattttaattagtttataatattatttaattaattaattaatatatttatataacattaatttatatattttatttaattaatatataattaatttattttatttaattaattaattatttaatttatttaattaattatttattttattttattaattaattatttattttattaattaattatttattttattaattaattatctattttattaattaattatctattttattaattaattatttattttatttaatttatttaattaattaatttatttattttatttaatttatttaattaattaatttatttattttatttaataatatgtttatttaacttattcattaatttattttaattaattatttaattaatattatatatatatattttttttaattaattaattaatttatttatttattttatttaattaatatgtttatttatcttatttattattattttaatatattatttatatatatattattttatttatttaattacttaatttatcttatttaattaatttattttatttatttaattattttattattttatttatttaattaatttattttatttatttatttatttctttatttaattatcttatttaattaatatatttatttattttattaagtaatgtatttttattatttatttaattaatatattatttatatataatttatttatttaattaattatttattaattttatatattattttatttataataaattaatttatatataattatttaacatatttattttattttatattaatttattaaaattatattatttaataaaatttttattatatattaattttatttaattattaaaaataatttaaaataataataataaaatatattcttttaattaaaataaataatataatagtatatttttaatgtgtaataattttaatatataattaatataatataattataaaaaatatatatttaatattaataaattaatttaatatttagaagaatattacatttatatatattttctataaaataattaaaatataatttttattatcaacaaatgatttatatatatatattatatatatgtgtgtgtattttaaattataatacttttttaattttatatatataaataagaaaattttacgagaaacatattttttagttttataattgtaatactattgtcttttctatgaaatatatattatat

SEQ ID NO: 11 - DNA, 2701 bp, Plasmodium falciparum
aataataaatcatatcaataagtaaaacataaaatattcagcattatatttattaattaaattaaatggtaataattcttataatatttatttaaagtaaataaatatttagtattaaatattataaaaaaaatattttaattatttaataaataattaatttattttaattattatataaaaaaaatatatatattatatatatttttaaaattaattaatatatattatttattatttataataataataaattatataaaattaaatattatatatatatttaatttaattaaataaaatatatattatttataataataataaattatataaaaatatatattatttatatatttaatttaattaaataaaatatatattatttataataataataaattaaataaaattatatattatttatatatttaatttaattaaataaaatatatattatttataataataataaattatataaaaatatatattatatatatatttaatttaattaaataaaatatattttatttataataataataaaaaatatatataaatatatatatatttaatttaattaattaatatatattatttattatatatttaataaaataaatatttagttattttatttgtaattaattatataataataaatatatatatatatatatatttttaattaaattaattaatatatgttatttattatatatacaataaataaatatttattttattaaatatataataaataatatatattaattaattaaattaaataatatatatatattaattaaattcaattatttaaatattatattattatttaaattaataattaattaatttattgtctaaataataataatatatatatataattatattaattatgtaaatttaaattataaatataaaaacataaaatattattaattaaataaaataaatatatttaataattaattaatttatttatttaaaaaaatatatataatatatattttatttatattaataaatatataaaataaaaaaaataaatatttttaatttatttatatttaattattaataaatatataattattttttaattaattatttaaatataaatataataaatttaattaaaattaattattattaaataatatatatatttaaaataaaatattaatttaatttatataatatttattattataatatattttatataaaatataattatttaattattatattataattaaaatataaataataatataaatataaatatataaaataatatatattactattatattcttattaatttatttaattaattatttttaattaaatatattattaaataattatatttattaatataaaaaacaattaattaattattatgtgtattataaaaataatatatatataatatattctgtaatttaattttaaaattataatttaatattattaaaataataaaatacaatttatatttatttataattaaataattaatttagtttaatttattaattaattaaataaaatatatatatatttatctattaaataattaatttaatttaattatattaattaattaaataaaatatatatatatttatatattaaataattaaaatttaatttaattatatgaattaattaattaattaaatatatataataatttatttatttataactattttaaatttaataaatataaaataaatataatattttaatttaaataaatattaattttatattaaatacgtaaattattttattaataaataaataaattaattaattaatttaattaaaatattaaaaaaatgtcttattatatatatatatttttaatttaataataatattatttattatttagtagtattaagttaattaataataaaataatactattttaaaatatttattattatttttttaaatttatatgtcttttaattaaaatattaatttattttatttaaattaattatatatattatatttattttaattaatatatttatattttattaaattaatatttatatttattatttatatatttttttattatttttatacatatatttaattaattaatattaatatttttattattttattaattaaattaaaattatatttattattattaattatatattttttatgtaaataaatatatatattttatttaattcttatttttattaaattaattttatatttaaattattatataatatttttatttatataaaattatataataatattattattaattatatattttattttaattaattaatatattattaataaatattattattattaattaataatttaaatatataaataaattaaattatatttcaattaaaaaaataaatatattattattgattattatttattattttttaatttacacattaaaattaactttattttttaaaattattatttttaatttaattatattattatttttttttatatataaatgaaatttatatattggattatagatattattaattttgtattttaattatatatttttttttaagaaatatatattactatattttttcattttgcaatattaactaatatattaatgtttgtcttattttaattatataaattttag

SEQ ID NO: 12 - DNA, 2800 bp, Plasmodium falciparum
cgttttcagtattagaaacatatacatattaactctataaataatgaagtgtaggtaacatataaacaatattctaattatatgaatttatttcgattcaaaatatactttttcatataatatttcattatttatatatgtttcataagaaaaaagaaataaatcttaataaataattttttttgatagattttctaggataatatgaaatatttcgtaaaaaaaataataataaaaatgatatttaaatatttatattaactacaatattaatattttattatttataaattatttatttaaaaatatataaatattaatttatggaatttttaaattaatttaaattaattgtttatatttaattatatatttaataaaatatattttaaaataatacacacaaaatgattcttaattaaatataaaatatttattttatttataatgaaataaataatttaatttaaaataaaataaaataaaaataataatatttattattatataatatatttaattaaattaaatttattatttaattaatttaaaataaaataaaaataataatatttattattatataatatatttaattaaattaaatttattatttaattaattaatttaaaataaaataaaaataataatatttattattatataatatatttaattaaattaaatttattatttaattaatttaaaataaataataattaaattaatatatattataattaattaaaaataaaataatattcaattataaatttattaataattttaaataaataattaaaataataataaattaaataatttaattaatataaataaacattataaattaaaaaataatttaataaatatatatataattattaataaatttaaatacatatatttttaataataatttcttaatttattttattacattattataatatatattttttatttaaaaaaatataattaaataaattaatttaataattaaatatatttaatagtaattaaatattaaacaaataatataaatattatataatattattaattaaattaaaataataatttattttataattatatatatatattaataattaatttaaaatataaattaagaaagataattttatacttttatttaattaaatatatagtaataaataattttatgttatttattataataatatttattattttattttatttatttaataaataattaattttataaaatatatttattttaaattaaaatataaacatataattaattaattaaatatatatatattttttttaatataataaataatatatttcacattttaattaaaataaaaataaccatttattaattaacttaattaatatataaaataaaataatttaattgtgtaattaaattaaatataaaacatttattaataattaattatatataatattatatattatcttaaattaattaatttttttaattattttaatataataattatttattaatattaattatgtatattttatttaattgtttaatatttattattattttatttaatatattattaatttaattaattattatattatatttaattattatattatttataatatattattaatttaattattgtttatttattatattatatattattatttaattattattttatttattatattatatattattaatttatataatataatttaattatatatatatttaatttatttatatattaatttaattatatatttatttaatttaattatatattttatttattaatttattttatttattaatttaatttaattatatatatatttaatttaattatatatatatttaatttaattatatatatatttaatttaattgtatatatatttaatttatttatatatttatttaatttatttatatatttaattatatatttatttttattttttataactataaattattaatttaattattaattttattttatttactaaatatataattaatttatatatattattgttttaattatttaaataattcattttatttaattaatttatatattattattaataattctttaatttataataattaattgtttaatatatattattatatttaattatttaaatattgttaattaattaatttattatattattatttaattaatttattatattattatttaattaatttatattaatattttattatttaattttaattaaaatttatttattattattttattttatattaatattagtattatttttttattaatttatattttaaatttaattatattaatttattatatattattaattaatttatattaatattttattatttataaattataatatttttatttatttaatatttaaattgtattaatatttttctatttattatttatattaatattcatattttattaattaatgtgtattatgaatttaattatattaatatttaaatttattattatttttatttattaaataattaacttaattatttattaatatatatatatttaattttaatatattttaaataaattttataatgatttaatttgatatattatataattatatatatatatttttatttattttataaattaataaatatatattatattaatatgtgattattatatttatatatttgtatagtcttatacatatataatatatatttgggggtatatatttggaatgttttttattattataa

SEQ ID NO: 13 - DNA, 2656 bp, Plasmodium falciparum
aaatatatataaatattatattatggtttatacacatttataaataacataaaaatttataaataaatacatataaatgaacagaatttatataagaattattatggaataatataagtattatagtatattaattaattaaaagaatacttatatataaatatattactaaaataaataaaaattaattatactttcataataatattattaatatttaaaaatacataattaaaaataaataaataataattaaaataatattaatataaaataaattataatataaaaaatatatattaattattaaattaatattagaatttaattaaattaaattaatataaaataaattataatataaaaaatatatattaattattaaattaatattagaatttaattaaattaaattaatataaaataaataatattatatataataaactatattttaagtcttaaattaatattataatttaattaaataaacaattaattgaaaataataatatattaattagttatttaaagtaaataaaatatataatttaattatactttattaataaataatattttataattatatatactgtaaattattacaataaataaaaaatatattaataaataatattattaaattaatactaaattaaataaaaataattaataaaaaaaattgtttattaattattaatatataaaataatcaattaattaatgattaaaaataaataaattaattaattatattaatataattaaatgttaaaataaatacattaattaataatataaatataattaaatttaaaataaataaattaattaataatattaatataattaaatattaaaataaataaataaattaattaattatttaaatataattaataatattatattaaatattaaaataaataaattaattaattaattatttaaatataattaataatattatattaaatattaaaataaataaattaattatttatttaaatataattaaataaattaataatattaatataattaaataaattaataatattaatataattaaataaattaataatattaatataattaaataaattaataacattaatataattaaatattaaaatagtaaattaattatttaaatataattaataatattatattaaataattaaatattaaaataagtaaattaattatataaatataattaatattattaaataataattaaataaatatattaattgtataaatattattaaatattaaattaattattaaaataaataaatttattcattatatattttttttactaaattaaatatataaataaagttatattaattaattaaataagacatttaaattaattattatttatttattgataaataaaataatttatttaattattatatattaattatatacattaaattatttaataaataatttttttattttataaatatatattaaatatttaattaaaattaaatataataaataaaaaagaattaaatatattaagtattaatatataattattattttatatttatatatattattattaaatatataattaaattaattattttattatattatttataaaatagaaaaaatatacacacacttattaaataaaaataaataatattaattaattaaaataatattaaattttattataaattaataatttattttaattttaattaaaatgacatatattaattaatttattaatatatttatatttttatatttaattaaacaatttaataaaataataatataattattatatatttatatatatattttttattatttaaataataaatatatatatattaataaattaattatatattaaattaataatttaaaaataaattatttattaattttatattttatttaataaattaaattataattaataaataatataaaaataaattattaataaaatatataataaattaattaaaataaataattttaaaaaataataaatatatattatatttttttatataataattttatttatattaaaataaataattatattatagtaaataaactttatatattattaataaattaattaattaattaatttaatattaaataataatatttaaaatatttattttagaataatatatataaatattaattataattattttatataatattttaaattaaattattttaatttatttaatattaatttaattaatacaatataaatatatatatatataaataataatgaatattttttataataaaattaattttatttatttataaaataattatttattataatattatttctaattttaaatgtttttataataatatatttaattaaaaagttaaaataaaaataatttcaataaatatattaatataatataattttattttataaaaaaaaaaattttaaataaaatagtttctaaataattaagcttttatttttaattattttttttttatctttatataatttaattatatatttatgaattctgtgtatttcttttgttaatatattttaaattaattataatattacaaatattttatatatttctataggaaacaaatta

SEQ ID NO: 14 - DNA, 2809 bp, Plasmodium falciparum
  misc_feature (1885)..(1886): n is a, c, g, or t
acaattaaaaaaatatatcataattttcaaaagacacatttgtttttaattaaatattattaaattaaaaattttgtatttcaaaataatacacatttggaaaaaaagacatgttactaatttatttaataataaaacatttataatgattaatttttaaatatatatataattattatatatatatataatattttaataaaataataaaaattatatttaattattaatttcaatatagtaatatatattatttatttatattcttatatattaatatgtatattaatattattaattttaattttaatataataattaatatttaataattaattaaaattaataattataaattattatttaatattatataatataatattaattaatataaaatattattatttaattattaattaatttaatttaataaattataaattaaataataattaatatttaataattaattaaatttaataaatataaattattatttaatattaaataatataatattaataaatataaaatattattatttaattattaattaatttattttaataaatataaattaaataataattaatttttaataaatatattataaatattaattattatataatttaaaaatataattcatcgttaattaattaaatttatatatttattatttaatttatatttttaatatattataaataattattaattattatattttaaattaaattatttatttaaataattataaattaataaattataatatattttttaattacttataatatttaataatataggaatattattgatttttttatatataatattatctttattaatttaataaataacttaatttgtaataatttaattttttaatattattttattattattaagtaaaataataaatatatttaattaaattatatttatttaattataattaaaaaatacactatttattattatattattgattaattaattaattaattaatatatttcagttaaattaattaaatatatttaatataaatataaagtattatataattatttattttaatagtaatatatttaataaaataattaatatttaattaaattaaattaaatatatttatttaatactaaataattaatatataataatatattatttatttaatattaatttcatttaaataaataattatatatttttattatactataaaagtttaatttattttattaatttaaatattataataaattaaagacatacataatataataataaattaataattaattaattatatataattattatttaattattattttattttaaatatattaatttaataattaatttaatattattttaaataaatttaatatctatataatttttattaaattaattttattattatttttttataaatgaatatattaattattataatatatatattattattatttatttataataataattattaattaatttatatattatttattttaattaaattagttaattatttatttttaatatattaattgttattgtgtatgtaacgaaatttatttataattaaattaattaattattttaatatgttaatttattaaatataaaattaatatttattttataattaaattaatgaattatttatttttatatattaattattatatgtccaattaatattatttatttataattaaattaattcatttattatatatataaattattattattgtaatttatattaattttatttatttaatattatttttatttattttattttattattttaattaattaattttatttatttaatattatttttatttattnnattttattattttaattaattaattttatttatttaatattatttttatttattatattttattattttaattaattaattttatttatttaataatatttttattattaatattattattatttaatatataattgattttatttattatattattattattattttatataattaattttatttattaattattattattttatataattaaatttatttattatattattattattttatataattaaattaatatatttaatattatatttaattattatattattttatataattaaatttatatatttaatattatatttatttattatattattttattattttattatataattatataattaatttaatttattatattattattttatattattaaatattaaatttatatatttaatattatatttatttattatattattttatataattaaatttatatatttaatattatatttatttattgtattattattattttataaattaattttatttattatattattattttatataattaaatttatatattttatattatatttattatattttattaatttatatatttaattttatttatttagtatttactattattattaaattaaatatttgtatttaatatattttattaatttataaatatatataatactttaaataactaatattgtaattaatattatacttcaaattaaattaataaatataattattatttaatataatgagtttatttctttttgttaaaatataatattattttttgtttttgaataaaatattcacaaaataaattttatacgtgttaaattttatatataataaaaaatgaaaactttaatttattcatgtacatttcattagacataatatgaaaacatatt

SEQ ID NO: 15 - DNA, 7850 bp, Artificial Sequence
  artificial chromosome
tcgaggtcgagggaaaatatacgtaatattttgttggtgagcttaaaaacttttaatattgtttgtgcaaaatttttgctaagaaaataattttatgagtcatattagaagttcattgaatttgttatataaaataaattttatattctgcctattttatgtacaacaaattgcggaattgtacataatataatatgaaaaatatatggctttaaattataattgccaaataaaatagggaggggccccccccaagccctcaaagggtgcatactatgtttataatatattattatatagcattatattagcattatatatatttgttattttacaatacattcattttgcgtggggcaatgcatttgcgctctttttcagttcatatttttgcaaataattgcactttcggaaatttgaaaaaatataaaacccataaaaaggtggaatacaaaacattttttgcattttcgaagcatataaatatgtatatatattatgcatactatattatgactatccttgccaataaaaaaaaaatattatataaaggggaccccatatataggtatatataatagctgctttaactaaggggctaataaatggccccgtattttgttttttttatagctaataataaaaataaaaaaataaaaagtacgaactttagtcccataaattaagaagtatatatgcttataaaaaggatatcaaataaattaatgttaactcaaataaattaattttcatgcaaaaaaatatttaaaaaagaatatatatattaagaaaaataattataaatattatttacatatatataccataatatatacccccttttgattatatatatataatattatatatttttctttagagaaaaaataaatagtgttatttcctattatatatcgtagttaattttaataatttacaatacactatatttcttattttatataaaatatatttataattaatttttttatattatatgtatatattttaattgcattctccttttttaataaaaattttacccaattgttatagagcttgttattattatataataacaatataagaatattttttatataacattaaaaaaaattaacatattttttattttttttaaatatttattccaatttatttttaacggtcaaattaaagaaatatatagatatatatttattgcttaaattataagtatatgtttttttaaaaaattaaagcccaaaacatatttctttgtattatatacatttttgcattttacactattttgccataagcacattaaatatatatatatatgcttgtagcaaattaattaaataaaataattaccaacattattatcccaataaattacaattacaattaaaaaaagatggctaacgcaaaagctagcagtaaaggagaagaattatttactggagttgtcccaattcttgttgaattagatggtgatgttaatgggcacaaattttctgtcagtggagagggtgaaggtgatgcaacatacggaaaacttacccttaaatttatttgtactactggaaaattacctgttccatggccaacattagttactactttaacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaagatcttcgtttttcttatttatatatttataccaattgattgtatttataactgtaaaaatgtgtatgttgtgtgcatatttttttttgtgcatgcacatgcatgtaaatagctaaaattatgaacattttattttttgttcagaaaaaaaaaactttacacacataaaatggctagtatgaatagccatattttatataaattaaatcctatgaatttatgaccatattaaaaatttagatatttatggaacataatatgtttgaaacaataagacaaaattattattattattattatttttactgttataattatgttgtctcttcaatgattcataaatagttggacttgatttttaaaatgtttataatatgattagcatagttaaataaaaaaagttgaaaaattaaaaaaaaacatataaacacaaatgatgttttttccttcaatttcgatgggggatcgtcgacggtatcgataagcttgggccctgaccatgattacgaaatcccggttcagggttcagggtttagggttcagggtgttaggtttagggtttagggttaggtttaggttttagggttcagggtttagggttcagggttcagggttcagggtttagggttcagggttcagggttcagggttcagggttcagggttcagggtttagggtttagggttcagggttcagggtttagggttcagggtttagggttcagggttcagggttcagggtttaaacgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggtttaaaccctaaaccctgaaccctgaaccctgaaccctaaaccctgaaccctaaaccctgaaccctgaaccctaaaccctaaaccctgaaccctgaaccctgaaccctaaaccctgaaccctaaaacctaaacctaaccctaaaccctaaacctaacaccctgaaccctaaaccctgaaccctgaaccgggatttcgttatcatggtcagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagacaaacaaaatgattatgatacaaataccttaaaataattttgatatattaattataaatatatgtaaaaactcacatataattttaattaaaaaataacaattatcaaaacataatagatatggtcaattaaatgttaataatacatttttaaatactataaaattaatataaataatatttatgtagtgaataacatttaaatatttaaaataataaattaaaataaataataaaattattgttgtaattaataaatatttaaatatattatataataaatattataattattattaaaataataaatggaaaattaacaatttattaatatatatatttttattaatatatatttttttattaaaataaatattaaagttttaaaataataataaaatattaatattaaaaaattaaataatattttaaaacataaattaattaatataaataattaagttaaaataagattaatttatttattaaatgtaatataattaaaatatttaatataataattatcgttaattatttaaaataattttaaattttataattaaatgtaatttatattttattatttttaaatagtatataattaatatatattaaattaatataaattacacacacaatataataaatttaatatattattttattatttataataattaaaataaataattaattatttaaaaatattaatgttaaaatataatatttacattatttaaaaaataaatattatttagtatactaattattattttatttttaatatattaatatcaataagaaattattcataattcatgttttaaatatgaaaattattatatattatttattaatttatattaaaatattaatattaattaatatattattttatatttaatattatattttatatttaattaattaaacgataataataattattaattgatatataagctatatattattatttaattaatatattaattattatattagtttatatttaaaatattatattattaattattatttaattattaattttaatatataaattatttatattaaatattattatttaattaatatattaattattatattagtttatatttaaaatattatattattaattattatttaattattaattttaatatataaattatttatattaaatattattatttaattagtatattaattattatattagtttatatttaaaatattatattatattaaataattatatatataatattttttataattatattttatatgtcttattgttataaaataaattctacagtaatatattaaaatactaaacatattttcttatttcttacaaaatgtaacccatcac

SEQ ID NO: 16 - DNA, 2062 bp, Plasmodium vivax
attaattaaattaaatatataattaattgaattaaattaatatattttaataataactgtctaataaattaaaataataattgaagctccataaattaatttaataaaaaaaatatatataaattattatataaataagattaaaataataaataaataaataataatataatatattaaataaataaaattaaaataataataaaatatattaaattaataaaataaataaatataaaaattagaatatttaattaaataaattaataaattaaatatattaaattaataaagtgaataaatataataatataaaaataaaaatatttaattattaaataaattaataaaataaatatattaaataaaaataatatatatatttattaattaacaaatatattattgttaataaaataaataaaaaaaataataaatatattaaataataattaaattaagtcataatataaattaaattgataaaattaacaaaataaatattaataataaaaataatattatataaaaaaaaatatataccatattattattaataatttctttaatatatttaaatataaaaatatattataataatttataattaaaataaatatataaatttattattaaaatatttatttaattaaataaataaattatattaaaatatttatttaattaaataaataaattattattaaaatataattaaaaatatattataaattaatatgtacataaaattttaaagttaaatatataattatattgaaatataattaatatgtatataataatatatacaataaataaattaatatatataataaatgaattaatttaatatacagttatattaatgcattaaataaattaataaataaatattttaattaatatattaataaataataatttatataatattaataaaataatattatttattatttaattaaataaataatttaaaataataatttatatttttaagttataacaaatatatttattaaaataaaataataatatatatttattgttatttaattaataaatatattttatttatatatattatcaaaataaataataatattattaatattttaaattatgtatataaaaacaattaaataaaaatataatttaataaatataaataaataataattaaaataaattaatgtgtatatatttattaaatataaatttaataattaattattttaattaaataaaattaaaaaataatatattaataaatacataatataataattaactaattattgtataaattaatttaatattaaataaataattaaaaaaagttatatatatatataatattaagaaaatacacaaattaaaacattaaaataattaaattaaattaatataatatatattttttatgtataattaattaattatatatttttatttaattaattatttaataattatttttacaaattaaattaataataatattaaaatgaatattatatatataaataaattaatttatattaaaataaaaataaataattcaactattaattaataatattataattaataaattattaaaccaaaattaacattttataattaataatattaattgattaattaaataatatatttttattatttatttaataataataattttaataaataatataataattaattttaataaataaattatatttattggaaattaaataatatattaattttaataatataaataataattaactttaataatttaattaatttttgtaaattaaattaaataaatttttaataaatcatttgttatattattatttagctaattatattaatattaataatttattgaataaataagaattaaataatttattttttaattaaataatattattaaataattaatttaaaataaatatattttatattataataaattaatgtgataataaaaattaaatatattaattatttataattaatttaatttatttattatataaaatataatataaataatacttat

SEQ ID NO: 17 - DNA, 2386 bp, Plasmodium vivax
aattataattaattatatttataatatggaaaatagcaatacttaaattatatattcataaaaaaaaatataatatattaaatagttaaattaattatttcatagaattaaaagataataatatataaaatttgtttaaattaattattatgattaattataatatttaataattataatttaaatattattttataattaattaaataaaattattaatatgtataattaaattaattaaaataatattttattcattcaattataggaaattgttttattaatttaattaattatatttatataagtgcttaaattgaaaaatatttaaattaattaatatattttaatatgacaatttaattaattcacaaagtaatataacaatttatttaaattaaactaaattaatatttatttaatataaatatttaattatttaataaatataaacatattaattaatattttatttaatataaaataaattattttaaaatataaatatacttaaaattaattaattaataaaataaattatttaatataaaattaattatttaatataaaataaataaatacaattaattatttaataaaataaataaattattttaatatataatacttaataatttaatataaggttaatcatttaaatatcatgaataataatttatttattttattaaataattaattaatttatttaatatttaaattatacaaacaaatatattaatcattatttacaatttaataaaataataaatataattatttatttaaaaaatattattttagttatttttaataataaataataaaaggtattttaatattataaatttaatttatataatattataataatttatttaaataataaaaataaattttattttatatatttttaacacaataaatatattaatatatttaaattaatttattttatttattatatatatatatatatatagtgattatttaaaaatatattaaatgtatatatttatttaatttattaaacagtttttaatattattattaaatatgtgatgtatttataattaattaataaaggattaaatttaatttaattaaaataattaattctgtgtattaataaataatatttatatatattaatatagttaaatagtgctattatttatttttaattcacttattatttttctttctactatattatatagttaattaatttatttataattatttaattaattaaatttatttattatttattaattaattaaaatttatttattattatttaattaaatttattatttaattattgctttattaatttattatttaattattaatttattaatttatgctaattaattaatttaatttaattattatattaaataattataattatatttattattttacttgttcatctttaaatgtatattactataatattttaatatattacatataatataattattttatatatatattaaccgttattataataaatatttttaataattcactttatataatatatttaacgattaattaaattattaatatttatatatattaaacatatttaatttagtaaaattaatattgcatatataattaattaatttatgtaacatattttaataattattatatttataattaattaatattttatttatatttaattaattaacattttatttattatatttaattaattaatattttatttaccatatttaattaatattttatttatttatatttaattaatttatatatttaatatgtatttaataattaattcgtataatatattttaaaattttatttaattaatatataatttatttgttaacgtatatttattttattatttaatataattaattaattatttatttaatatatttgatttatttattatttaacatatttaatttatttattatttaacatatttaatttatttatttaatatatttgatttatttattatttaacatatttaatttatttattatttaatatatttaatttatttattagttaatatatttaatttatttatttaatatatttaatttatttatttaatatatttaattaatttattgtttaacacatttaatataattaatttatttaaatacttatttgttatttaaaatataataaacgtttttgttaagtacaatatattaaaattattttatattttaaatttaattaatatataattactttatttaattttatttttatttaacttttataagttaatatttctttttatttaacataatttgttttatttatatatattttaactgtaattatacataaattatattcatttttaataaat

SEQ ID NO: 18 - DNA, 2080 bp, Plasmodium vivax
ctaaaaactatcattttataatttattaaatatatttatatatatttaatttgttaaaaattagttttatttaatacaaaattatttaataataattaaaatattaatattttattaaattaattaattataaaattattattttgtctaatatataattaaatattatatttataatatattaaattaaaataattaaatattttaattatgtataaaaatactcgtaatattaaatattatttaatattttatttaatttatttaatatgttaattatttataaaattattatttaatatatatattatttataaatttataatttaatatatattaacatttatttaatatataataattattatattattttgttttaattgtattaatttaattaattaattatcatatataatattatttattttaatttactattaattatttatttaatttattaattatattaattttgttttaatatatataaattatattaactattttattatttaaataatttattaaacataattattaattaattaattaattacaaataaatattatttaaattaatataattaaataatattaaatatttataataaaaaaaatatatattccttttatttaaataacattaatataataatatatttttaaattaaataattattatataataatttttaatatatttaatttaatactcattttaattattaaatttaatttattatttatatataaattaattattatatatatattaaaattattcgttttaatacagacattgtgttaataaaattaatttttattaaaattaatattaaaatataaaataaaaataaatgttaatataaatatatttatttaattaaataaaataaattaaatacatcttaaattttattattttaatataattattaaaattaatatataataatttaaaattatatattattttaattaattaattataattaaataataaattaatattttatttattattaagtgcaaatataattataattatgttattaaaataataaatattatatataatatatatatatttatacttattatttaaataaatttatatttatttaatatatttttttatttaatttataattttatttatttaatatatattatttatatttattaattaataattaatttattttaatatttaattatttttattttatattaatattactttaatttaattaattattatatatatttgtaattttattttaatattatacacttataaagtatgttaattaattttttatatgttatttataattaatttatatatttatttatattattttataatatgtaaattactttttataataattaataatattaatttaattatatattaattttaataagttaaattatatatatatatttattatttaaataatataatttaatatatttttaattaaatattatttaatgtgttatattttattaaattataaatttatattaatcatattaattgtaataatattttaaaaataatattatatatgctaataattattattgtttatttattaattaattttaaataatattaaattaaaattattatttttaatatattaatatttattatcatttaataattaataaaatatattataaatttaatatatgtttattcattttattgtttaattaaatatatttaatattatttagaattaaataattaatttaattacattattttatttttaattaatttatttattttaattaattaattagtttattttaattaataaattaataagtttatttaatttatttattttaacattttataatgtgtattaatattatgtttaatattttcactattaatatatttatttcaatattatattttattttttaaaataattttatttaatgagtttatttaaatattgtttattttattaattaattattatttttttttaataatttataatatataatcttattttaattttgttcaacgtttatatttatttattaa

SEQ ID NO: 19 - DNA, 1954 bp, Plasmodium vivax
aatatatatataatacacatattttaaataataaacaaattaaaataaaataaataactttatatataaataaaaattgaaatatattaatcaataaattattattaatatatattaaataataattaaaataattataaattaaatgaaataaaataaataaaaaataacaaaattaacgaataaattaaattaaataagtaaaagttaattaaaataaattaattattaaaaatattacttttagttattatttatatgtatataaatttaaatatataaattaataaatatataaattaaattaaatatattaaataaaatatgttatattaattatatataaatataaaataaataaaattattaaattaaaaagctaaattaattattaatataaataaaaatatatattataattaaattaaataaaaattaattttacatattaattatgattattatttaaaaaaaatatataaaatatttaaatatttattattaatataaatataaaataaatgtaagagcataaacaaaaatgataaattatttaaaatatatataatatattaaaaaataaataattaaaataaattaataattaaataattaattataaaaatataattaataaataatatatataagttaatttaaagggtaaataaaaattaataattatattattaattattatataaattaatataactatttaaaattagtattatataattaaatgttaaataaataagaaattaaatataaattaattaaattaataatatagtattaataattaaattaataaaaatagttacataaattatataatatattatttttattaatttaattataataattattttaaaataaataaataaatataattaaaataaatatattaataatataattaattaattaatatttatttaataattacaggggaaaataatatatataattaaaattataaatattaaaattaattatttgatgtaaattaaaaataataaataatataattaaattaattaatatatataacaatttaagttattatataataaataaaaagaattaattaataattaataataaaattaaataaattattttaaataataaataaatataatatatatgtttattaaatttaattcataattaattatataataatatatatttttatttatttaataaatttaattataaaatagtaatatttaattaaaatatacatataaaaacaaataattaaataattatttaatataataaataataattacacaataattaataattatatatatatatttaatacattatatttattttaaataaattaatttaaaaattaaaaatattaaataatgttttaagtataattaatgttataaattaattttaatatatataatatattatatattttataaaattaaaaatataatagttaaatataaataaattaacacaatttaaattataaatatataatatgtgaattgtttttttaataatataaaataaaatgtatttaataattaatttatatgtaatatattgtgtaataaatatttaattaaattattttaattaaaattaatttaaatacattaattaatttattataattgaataaataaataattaattattatattaaattaaaataattcaataattaactaatatatttaattatttttaaaaaattaattaattaattgattaattaaatatttaatttaacatattaaaataattaaataaatatataactattaaaataataaaaataaataaataaattaatatatattttataatatatatattaaattaatacataattaattatattatataaattaattaatatatatatttaaataaaatattaaaattaataatattaatttaattctttcgcatt

SEQ ID NO: 20 - DNA, 8011 bp, Artificial Sequence
  artificial chromosome
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacaaaataccaataataccgtttggagaattgaaagctctctagatccctccttttattttatgaggaacgttttgaaagacttgcataacaacatgtctatatgaccaactacaccgatcattttttaacttttttaacttttaattttttcacttttaatgtgcccccaaataaaaacttccctttgtaacattaacaaatgaaagcttgtatacatttcagagctaatgcatcaaaagtggtaaaagcttcgatcaaatgtggtggtcattttccactttggatgaaatgtgaaagtaatcatcttaattatgcttgcaaatgttaattatgttattttattttgaattatttatagttaaaaaaaaagacgcatattcataaaatattacacataacaaatatatgatatgcgattaccaaactatactgataaagtttgaacaaatacataagtattgttgatatttcttctatagtttaatgttaataataatattatagtattataaagtagtgatatacaagttgtagggtctagagtaattcgactccattcactttattcctttttcatatatacaacaaaaaggaggtacacaaatttatgtatttcgacacaaaaaatatatattttgttcgaaaaaatatgttcgtggcattccacattttaaaattaacagtacatatgtaaaaatactattcacaaaagaagtacagaacaataatagatctttatttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttcccgtcatctttgaaaaatatagttctttcctgtacataaccttcgggcatggcactcttgaaaaagtcatgccgtttcatatgatctgggtatcttgaaaagcattgaacaccataagttaaagtagtaactaatgttggccatggaacaggtaattttccagtagtacaaataaatttaagggtaagttttccgtatgttgcatcaccttcaccctctccactgacagaaaatttgtgcccattaacatcaccatctaattcaacaagaattgggacaactccagtaaataattcttctcctttactgctagcttttgcgttagccatttttataaaatttttatttatttataagcaaatatatatttttatatatttattttatattttgaaatatttttaaaatttaatttattttttattatatactttaaaaatggcagttattaaaaaataaggataaaattattattatttttaattggtgttttgtatttttttttgttatataatattaaattttaatttggagaaaatgtttattgtattcaatataataacataatatttatttcattttattttagaaatacacgtatttttttttcaactctatactatgtacttatgtgtataaattattaaaataaataaaaatattataaaatgcatataatgtagggataaaatattactatatttatagtgtcagtgcaggtgtaaaatcctcggagtataaaaaaataaaatggattatttaaaaaaaggtactgcataaaacatttaatttgatgcttgtagatgagttaagcttaattcttttcgagctctttatgcttaagtttacaatttaatattcatactttaagtattttttgtagtatcctagatattgtgctttaaatgctcacccctcaaagcaccagtaatattttcatccactgaaataccattaaattttcaaaaaaatactatgcatataatgttatacatataaacataaaacgccatgtaaatcaaaaaatatataaaaatatgtataaaaataaatatgcactaaatataagctaattatgcataaaaattaaagtgccctttattaactagtcgtaattatttatatttctatgttataaaaaaatcctcatataataatataattaatatatgtaatgttttttttattttataattttaatataaaataatatgtaaattaattcaaaaaataaatataattgttgtgaaacaaaaaacgtaattttttcatttgccttcaaaatttaaatttattttaatatttcctaaaatatatatactttgtgtataaatatataaaaatatatatttgcttataaataaataaaaattttataaaaatggttggttcgctaaactgcatcgtcgctgtgtcccagaacatgggcatcggcaagaacggggacctgccctggccaccgctcaggaatgaattcagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggtgcacattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgattagtcgagggatatggcagcttaatgttcgtttttcttatttatatatttataccaattgattgtatttataactgtaaaaatgtgtatgttgtgtgcatatttttttttgtgcatgcacatgcatgtaaatagctaaaattatgaacattttattttttgttcagaaaaaaaaaactttacacacataaaatggctagtatgaatagccatattttatataaattaaatcctatgaatttatgaccatattaaaaatttagatatttatggaacataatatgtttgaaacaataagacaaaattattattattattattatttttactgttataattatgttgtctcttcaatgattcataaatagttggacttgatttttaaaatgtttataatatgattagcatagttaaataaaaaaagttgaaaaattaaaaaaaaacatataaacacaaatgatgttttttccttcaatttcggatgggggatccactagtaacggccgccagtgtgctggaattcaattaaatattatttaattattaatatattaattatttagacttaatattatattgtatattataaaataataaatgaaatatattaattaatattatttaaaataataataaatttattaatatattataaattatattaaaaatataattatttaattaaattgtttaattttatttaatttattaaaataattattttattatatattaattaatataatttatcttaataataattatatatatatatatatttatttaatattatttattttaattaataaatatttaattaaatataaataaataatatataattaatttaatttattatttatttaattataaaataaataatatatatttaatatattattaaattaattaattaactaattataaatatttaataattaatattgtacaacaaatatatattttttaatttaattattattattaaattattatttttaaataataataaatatatatatatatatatatttatttatataatataataataatttataatttaataatattttaatttattaaataaaaagttaaattaattattatatatattatttttaattatttacacataattatatatatattatttaatattttattaatttaaattaattattattaagttaaaatttagatattgtattatttaatataattaaaataataaacattagataataattagtattatttattcaatatatattattttattacaaatatattattaatttaatttaaaatataatttattattttttatttaatataattaattaattaattatttattattaacaatttaaatattaattatatacaattatatataaaatatattaaatatttattatttaattaattaataattattattaattaattatttattatttatttaattaattatttattatttaattaaatatttattatttaattaaatatttattatttatttaattaattattattatttatgtatttaattaattattatttatttaattaattaatattattatttaattaattatttatttaattattattatttaattatttattattatttatgtatttaattaattattatttatttaattaatattattatttatttattattatttatttatttattattatttatttatttattattaattaattgtttattattatttatttatttaatttattattaacaatttaaatattaattatatatataattatttattttaatatattatatatttatttttaaattaattttatttatatatttaaaatatttatttaatttaataattattttattaaatataaattatattatttcttaattttattatttaaatgtaaaaataatatatatgtgtgtttataatatttaatttaattatacataaattattataagtattgggaattctgcagatatccatcacactggcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

(19) United States
(12) Patent Application Publication
(10) Pub. No.: US 2013/0078635 A1
(43) Pub. Date: Mar. 28, 2013

(54) RAPID METHOD FOR IDENTIFYING DRUG-RESISTANT GENE USING ARTIFICIAL CHROMOSOME OF PLASMODIUM, AND METHOD FOR PREPARING RECOMBINANT PLASMODIUM

(71) Applicants: Shiroh Iwanaga, Mie (JP); Masao Yuda, Mie (JP); Izumi Kaneko, Mie (JP)

(72) Inventors: Shiroh Iwanaga, Mie (JP); Masao Yuda, Mie (JP); Izumi Kaneko, Mie (JP)

(73) Assignee: MIE UNIVERSITY, Mie (JP)

(21) Appl. No.: 13/636,905

(22) PCT Filed: Mar. 25, 2011
(86) PCT No.: PCT/JP2011/001781

§ 371 Date: Dec. 10, 2012

(30) Foreign Application Priority Data
Mar. 26, 2010 (JP) 2010-071688

(51) Int. Cl. C12N 15/11; C12Q 1/68

(52) U.S. Cl. CPC C12N 15/11; C12Q 1/6806
USPC 435/6.11; 435/471

(57) ABSTRACT

The present invention relates to a rapid method for identifying a drug-resistant gene using an artificial chromosome of a protozoa. Specifically, it relates to a method for screening for a drug-resistant gene, which involves preparing a recombinant Plasmodium using an artificial chromosome of a Plasmodium, either inoculating the recombinant Plasmodium into a non-human mammal or using the in vitro culture system of the Plasmodium at the blood stage in the red blood cells, and using the survival of the recombinant Plasmodium in the presence of a drug as an index. The present invention also relates to a method for preparing a recombinant Plasmodium by directly introducing an exogenous gene into a Plasmodium at a high efficiency.

### TECHNICAL FIELD

This application claims priority based on Japanese Patent Application No. 2010-071688, filed on Mar. 26, 2010, the contents of which are incorporated herein by reference.

The present invention relates to a rapid method for identifying a drug-resistant gene using an artificial chromosome of a protozoa. Specifically, it relates to a method for screening for a drug-resistant gene, which involves preparing a recombinant Plasmodium using an artificial chromosome of a Plasmodium, either inoculating the recombinant Plasmodium into a non-human mammal or using an in vitro culture system of the Plasmodium at the blood stage using red blood cells, and using the survival of the recombinant Plasmodium in the presence of a drug as an index. The present invention also relates to a method for preparing a recombinant Plasmodium, which involves directly introducing an exogenous gene into a Plasmodium at a high efficiency.

### BACKGROUND ART

Malaria is transmitted by Anopheline mosquitoes and is a protozoal febrile disease occurring due to the infection of the red blood cells with Plasmodia belonging to the genus Plasmodium. Four Plasmodia, Plasmodium falciparum (Plasmodium falciparum), Plasmodium vivax (P. vivax), Plasmodium malariae (P. malariae), and Plasmodium ovale (P. ovale), are known as Plasmodia capable of infecting humans. Among these, falciparum malaria caused by Plasmodium falciparum is considered to be the most important in view of the number of patients, severity, and the spread of drug resistance. Plasmodia infect mammals other than humans, mainly primates and rodents through the medium of Anopheline mosquitoes. Two Plasmodia, Plasmodium knowlesi (P. knowlesi) and Plasmodium cynomolgi (P. cynomolgi), are known as a Plasmodium capable of infecting monkeys, and three Plasmodia, Plasmodium berghei, Plasmodium chabaudi, and Plasmodium yoelii, are known as a Plasmodium capable of infecting mice.

The development of a malaria vaccine is said to be difficult because of the change of the antigen of protozoas thereof and the like. In recent years, it has been attempted to develop vaccines against malaria by producing antigens of such a protozoa using a genetic engineering technique, or the like; however, they are not yet put to practical use. Thus, the treatment of malaria is now carried out by administering an anti-malarial drug such as chloroquine, quinine, pyrimethamine, mefloquine, primaquine, and artemisinin. However, the appearance of drug-resistant protozoas and the broadening distribution thereof make treatment using an anti-malarial drug difficult; thus, it becomes imperative to identify a drug-resistant gene, elucidate a resistance mechanism, and monitor the distribution of resistant protozoas.

The identification of a drug-resistant gene of a Plasmodium has conventionally been performed by crossing a drug-resistant protozoa and a wild-type protozoa and conducting polymorphism analysis (RFLP or the like) using a chromosomal marker of their offspring protozoas; however, the method requires a great deal of labor and time since transmitting mosquitoes are necessary for the crossing of the offspring protozoas and a large number of offspring protozoas are necessary for the analysis. In fact, only 2 to 3 drug-resistant genes have been identified for the past 20 years. Even when a candidate drug-resistant gene has been identified using the above method, it is necessary to prepare a genetically modified protozoa and confirm the resistance ability.

Gene introduction for preparation of a genetically modified Plasmodium falciparum is carried out using a method which involves introducing DNA into non-infected red blood cells and then infecting them with a protozoa for the natural incorporation of the protozoa (an indirect introduction method), or a method which involves performing the synchronous culture of protozoas to directly introduce DNA without purifying and recovering the protozoas at the time at which many ring-stage protozoas are present (a direct introduction method). These methods have extremely low gene introduction efficiency and should be improved in terms of time, cost, and labor since they require at least one month and, in prolonged cases, even at least a half-year to obtain a recombinant protozoa. In view of such necessity, use of schizont-stage Plasmodia has previously been believed to improve the gene introduction efficiency because the Plasmodia at the stage can infect a red blood cell immediately after gene introduction and many Plasmodia are present in the infected red blood cell. However, because electroporation causes the death thereof, they could not actually be used for the introduction of gene.

As described above, a method for rapidly and precisely identifying a drug-resistant gene has not been established despite that it becomes imperative to identify a drug-resistant gene of Plasmodia, elucidate a resistance mechanism, and monitor the distribution of resistant protozoas. There is a need for the development of a method for gene introduction into a Plasmodium falciparum more rapidly and more efficiently than conventional techniques, which can be used also for the identification of a drug-resistant gene.

### CITATION LIST

Non Patent Literature

Non Patent Literature 1
Wellems, T. E. et al.: Nature, 345: 253-255, 1990

Non Patent Literature 2
Wellems, T. E. et al.: Proc. Natl. Acad. Sci. USA, 88 (8): 3382-3386, 1991

Non Patent Literature 3
Su X. et al.: Cell, 91: 593-603, 1997

Non Patent Literature 4
Malariology Laboratory Manual [in Japanese] (Saikon Shuppan): 217-227, 2000

### SUMMARY OF INVENTION

### Technical Problem

An object of the present invention is to provide a rapid and precise method for identifying a drug-resistant gene of a protozoa and, specifically, to provide a rapid and precise method for identifying a drug-resistant gene of Plasmodia. Another object of the present invention is to provide a method for introducing a gene into a Plasmodium more efficiently than conventional techniques.

### Solution to Problem

In order to achieve the above object, whether a candidate gene is a drug-resistant gene or not has been determined by preparing a genetically modified Plasmodium into which candidate gene fragments are introduced, inoculating it into a non-human mammal or culturing it in vitro in the presence of red blood cells to infect the red blood cells, and using the survival of the recombinant Plasmodium in the animal body or the culture system after drug administration as an index. The artificial chromosome introduced into a Plasmodium selected in such a way has been recovered, and the nucleotide sequence of the candidate gene fragment incorporated therein has been determined to identify the drug-resistant gene. For the introduction of candidate gene fragments, an artificial chromosome of a Plasmodium independently developed by the inventors (Japanese Patent Application No. 2009-051454), which enables the rapid and simple gene recombination in a Plasmodium, has been used. The use of this artificial chromosome of a Plasmodium has enabled a drug-resistant gene to be identified more rapidly than conventional methods.

In preparing a genetically modified Plasmodium falciparum into which candidate gene fragments are introduced, a schizont-stage Plasmodium at a stage immediately before entry into red blood cells is prepared by synchronizing the cell cycle of Plasmodium falciparum, followed by directly introducing a gene into the Plasmodium at the particular cell cycle stage, which has enabled the genetically modified Plasmodium falciparum to be obtained in a shorter period of time than conventional methods.

Thus, the present invention relates to a method for screening for a drug-resistant gene, comprising the steps of:

(a) preparing a recombinant protozoa into which an artificial chromosome containing candidate gene fragments is introduced;

(b) inoculating the recombinant protozoa into a non-human mammal, followed by administering a drug, or culturing the recombinant protozoa in vitro and adding the drug to the culture system; and

(c) recovering a drug-resistant recombinant protozoa from the non-human mammal or the culture system and identifying a candidate gene fragment contained in the protozoa as a drug-resistant gene,

wherein the artificial chromosome is an artificial chromosome of a protozoa containing a protozoa-derived centromere region.

The present invention also relates to the method for screening for a drug-resistant gene, comprising the steps of:

(a) preparing a recombinant Plasmodium parasite into which an artificial chromosome containing candidate gene fragments is introduced;

(b) inoculating the recombinant Plasmodium parasite into a non-human mammal, followed by administering a drug; and

(c) recovering a drug-resistant recombinant Plasmodium parasite from the non-human mammal and identifying a candidate gene fragment contained in the Plasmodium parasite as a drug-resistant gene, wherein the artificial chromosome is an artificial chromosome of a Plasmodium parasite containing a Plasmodium parasite-derived centromere region.

The present invention also relates to the method for screening for a drug-resistant gene, comprising the steps of:

(a) preparing a recombinant Plasmodium parasite into which an artificial chromosome containing candidate gene fragments is introduced;

(b) culturing the recombinant Plasmodium parasite in vitro using red blood cells to infect the red blood cells therewith, followed by adding a drug to the culture system; and

(c) recovering a drug-resistant recombinant Plasmodium parasite from the culture system and identifying a candidate gene fragment contained in the Plasmodium parasite as a drug-resistant gene, wherein the artificial chromosome is an artificial chromosome of a Plasmodium parasite containing a Plasmodium parasite-derived centromere region.

The non-human mammal may be a rodent or a primate.

The artificial chromosome of a Plasmodium parasite may be one containing a centromere region derived from Plasmodium berghei, Plasmodium falciparum or Plasmodium vivax.

The artificial chromosome of a Plasmodium parasite may be a circular artificial chromosome or a linear artificial chromosome.

The candidate gene fragments may be gene fragments derived from a drug-resistant Plasmodium parasite.

The method of the present invention may be performed even when the candidate gene fragments have an average length of 2.5 to 4.0 kb, 4 to 10 kb, or 10 to 50 kb.

The drug may be a therapeutic drug for malaria selected from the group consisting of for example, chloroquine, quinine, pyrimethamine, mefloquine, primaquine, and artemisinin.

The present invention further relates to a method for preparing a recombinant Plasmodium parasite, comprising the steps of:

(a) preparing a schizont-stage Plasmodium parasite at a stage immediately before entry into red blood cells; and

(b) directly introducing an artificial chromosome into the schizont-stage Plasmodium parasite at the stage immediately before entry into red blood cells by electroporation, wherein the artificial chromosome is an artificial chromosome of a Plasmodium parasite containing a Plasmodium parasite-derived centromere region.

The Plasmodium parasite may be selected from the group consisting of Plasmodium falciparum, Plasmodium vivax, Plasmodium malariae, Plasmodium ovale, and Simian malaria parasite (P. cynomolgi and P. knowlesi).

The method for screening for a drug-resistant gene may be performed by preparing a recombinant Plasmodium parasite into which an artificial chromosome containing candidate gene fragments is introduced using the method for preparing a recombinant Plasmodium parasite.

Here, the Plasmodium parasite may be selected from the group consisting of Plasmodium falciparum, Plasmodium vivax, Plasmodium malariae, Plasmodium ovale, and Simian malaria parasite (P. cynomolgi and P. knowlesi).

### Advantageous Effects of Invention

The use of the identification method according to the present invention enables the completion of all the steps required until the identification of a drug-resistant gene in the order of 1 to 3 weeks, enabling the dramatically rapid and simple identification of the drug-resistant gene compared to that for conventional methods. According to the identification method of the present invention, a drug-resistant gene can be reliably identified because the selection thereof is performed using the survival of a recombinant Plasmodium in the presence of the drug as an index, that is, the Plasmodium actually exhibiting drug resistance is selected. In addition, the use of the method of the present invention enables the identification of a plurality of drug-resistant genes at a time.

The use of the method for preparing a recombinant Plasmodium according to the present invention also enables the efficiency of introduction of an exogenous gene to be improved by about 1,000 or more times over that for conventional methods; thus, the recombinant Plasmodium can be obtained in a shorter period of time than before. Therefore, a drug-resistant gene can be more rapidly and more simply identified when the method for screening for a drug-resistant gene according to the present invention is carried out using the method for preparing a recombinant Plasmodium according to the present invention.

### BRIEF DESCRIPTION OF DRAWINGS

FIG. 1 is a diagram showing the schematic of a method for screening for a drug-resistant gene of a Plasmodium.

FIG. 2 is a schematic diagram showing a structure of an artificial chromosome of a Plasmodium berghei.

FIG. 3 is a photograph showing results of PCR indicating that a recombinant Plasmodium selected according to the identification method of the present invention has a drug-resistant gene.

FIG. 4 is a photograph showing results of southern blot analysis indicating that a Plasmodium selected according to the method of the present invention has a drug-resistant gene and that a plurality of types of DNA fragments containing a drug-resistant gene is incorporated in an artificial chromosome, indicating that a plurality of types of drug-resistant genes can theoretically be identified at a time.

FIG. 5A is a photograph showing results of southern blot analysis indicating that a Plasmodium selected according to the method of the present invention has an artificial chromosome (gfp was used as a probe).

FIG. 5B is a photograph showing results of southern blot analysis indicating that a drug-resistant gene is incorporated in an artificial chromosome in a Plasmodium selected according to the method of the present invention (human dhfr was used as a probe).

FIG. 6 is a diagram showing the schematic of a method for screening for a drug-resistant gene of a Plasmodium falciparum.

FIG. 7 is a schematic diagram showing a structure of an artificial chromosome of a Plasmodium falciparum.

FIG. 8 is a pair of microscopic appearances of a schizont-stage Plasmodium at a stage immediately before entry into a red blood cell for a Plasmodium falciparum (a bright-field image and a fluorescently detected image (nuclear staining)).

FIG. 9 is a graph on the percentage of parasitism of Plasmodium falciparum and the number of days after gene introduction when the introduction was performed according to a direct introduction method and an indirect introduction method (an artificial chromosome or a control plasmid was used).

FIG. 10 is a photograph showing results of southern blot analysis indicating that a gene library of a Plasmodium falciparum was constructed.

### DESCRIPTION OF EMBODIMENTS

The present invention relates to a method for screening for a drug-resistant gene using an artificial chromosome of a protozoa. Specifically, the present invention relates to a method for screening whether a candidate gene is a drug-resistant gene or not, which involves preparing a recombinant Plasmodium into which a candidate gene fragment is introduced using an artificial chromosome of a Plasmodium, inoculating a non-human mammal therewith, followed by administering a drug or performing culture using an in vitro culture system of the protozoa at the blood-stage in red blood cells, followed by adding the drug to the culture system, and using the survival of the recombinant Plasmodium in the presence of the drug as an index.

The present invention also relates to a method for preparing a genetically modified Plasmodium, which involves preparing a schizont-stage Plasmodium at a stage immediately before entry into red blood cells by synchronizing the cell cycle of the Plasmodium and directly introducing a gene into the Plasmodium at the particular cell cycle stage.

### 1. Definition

The term “drug-resistant gene” refers to a gene encoding a molecule affording resistance to a drug, such as a gene encoding an enzyme inactivating a drug. A therapeutic drug therefor becomes ineffective or less effective against an organism having obtained the resistance through the action of the molecule encoded by the gene.

The term “protozoa” refers to a eukaryotic unicellular animal, that is, a protozoan.

In the present invention, the term “recombinant Plasmodium” refers to a Plasmodium having an artificial chromosome of a Plasmodium into which a candidate gene fragment is introduced.

The term “centromere region” is a genomic region acting on the equipartition of a chromosome during cell division. In a protozoa of the genus Plasmodium, the centromere region is generally known to be rich in an adenine-thymidine base pair and have repeated sequences. According to the present invention, the sequence of the centromere region may be partially modified provided that it has a function as a centromere. Here, the “partially modified” refers to that one or a plurality of bases are substituted, deleted, inserted, and/or added in the sequence of the intended region.

The typical life cycle of a Plasmodium is as follows. A sporozoite is injected into a host through the blood-sucking of a mosquito. The sporozoite reaches the liver cells through the bloodstream, parasitizes them, and propagates. The propagated protozoa becomes a merozoite (liver merozoite), which can infect red blood cells, is released from liver cells into the bloodstream, and infects red blood cells. When the protozoa infects red blood cells, it differentiates into a ring, a trophozoite, and then a schizont, and again becomes a merozoite (red blood cell merozoite). After being released from red blood cells, the merozoite again infects red blood cells and repeats the above differentiation, while some protozoas, in the course of differentiation, differentiate into male and female gametocytes. When the blood is sucked by a mosquito, the gametocytes move to the midgut of the mosquito, become male and female gametes, and, after conjugation, differentiate into zygotes and then ookinetes. After differentiation, cysts (oocysts) are formed in the midgut, and sporozoites are formed in the cysts. The sporozoites then move from the midgut to the salivary gland and wait for the next blood-sucking, thereby resulting in the completion of the life cycle of the Plasmodium.

The schizont-stage Plasmodium can further be distinguished between an early schizont Plasmodium and a late schizont Plasmodium. Here, the schizont-stage Plasmodium generally refers to a Plasmodium “from when nuclear division starts until immediately before being released from red blood cells”, and at the schizont stage, the number of nuclei is known to increase to 2, to 4, and so on until reaching 32. The “merozoite-stage malaria protozoa” that is formed by differentiation of schizont-stage Plasmodium is known to be not contained in red blood cells or parasitophorous vacuoles (Michael J. Blackman: Cellular Microbiology, 10: 1925-1934, 2008).

Accordingly, for the purpose of the present invention, the “early schizont Plasmodium” is defined as a schizont-stage Plasmodium in which the number of nuclei is 2, 4, or 8, and the “late schizont Plasmodium”, as a schizont-stage Plasmodium in which the number of nuclei is 16 or 32.

For the purpose of the present invention, the “schizont-stage Plasmodium at a stage immediately before entry into red blood cells” or “mature schizont-stage Plasmodium” refers to a late schizont Plasmodium immediately before becoming a merozoite, and is defined as a Plasmodium in which the number of nuclei is 16 or 32 and either the red blood cell membrane or the parasitophorous vacuole membrane is lost.

For the purpose of the present invention, the “medium forming a density gradient” refers to a liquid or particulate substance forming a density gradient in itself or by centrifugation.

### 2. 1 Artificial Chromosome of Protozoa

According to the present invention, the artificial chromosome of a protozoa contains a host protozoa-derived centromere region and may further contain a telomere region and exogenous genes such as a gene encoding a marker protein and a reporter gene. The exogenous genes include genes encoding traceable marker proteins, for example, genes encoding a green fluorescent protein (GFP), a yellow fluorescent protein (YFP), and a cyan fluorescent protein (CFP), and other reporter genes. The artificial chromosome of a protozoa may further contain a control region which is a region necessary for expressing an exogenous gene or regulating the expression of the exogenous gene.

The base for the artificial chromosome of a protozoa may use, for example, a plasmid selected from pBR322, pUC18, pUC19, pUC118, pUC119, pBluescript (registered trade name), and the like using Escherichia coli as a host, and the sequence of a centromere region, the sequence of a telomere region, the sequence of a control region, and the sequence of an exogenous gene are incorporated in such a plasmid in an arrangement in which the artificial chromosome can function. The plasmid refers to an independent element used for the introduction of a nucleic acid into a cell.

The host protozoa may be, for example, a protozoa belonging to the phylum Apicomplexa such as protozoas of the genus Toxoplasma, the genus Babesia, and the genus Plasmodium, and a protozoa of the genus Trypanosoma. In the protozoa of the genus Plasmodium, for example, because the centromere region is generally known to be rich in an adenine-thymidine base pair and have repeated sequences, the centromere region can be deduced.

Additionally, in the protozoa of the genus Trypanosoma, the region rich in guanine-cytosine is presumed to be a centromere region (Genome Biol. 2007; 8(3): R37).

### 2. 2 Artificial Chromosome of Plasmodium

According to the present invention, the artificial chromosome of a Plasmodium can be used which was previously developed and filed for patent application by the inventors (Japanese Patent Application No. 2009-051454). The artificial chromosome of a Plasmodium has the advantages that:

(1) it enables the rapid preparation of a recombinant plasmodium parasite compared to conventional plasmids because it is uniformly distributed into daughter plasmodium parasites at the time of cell division owing to the action of the centromere, resulting in no production of a plasmodium parasites free of the artificial chromosome;

(2) it is always present in the nucleus independently from the plasmodium parasites chromosome without being incorporated into the chromosome by homologous recombination during DNA replication owing to the action of the centromere;

(3) it does not produce an abnormal replication which is seen in a conventional plasmid (for a Plasmodium, a typical plasmid produces a rolling circular replication during replication, resulting in causing the concatemerization of the plasmid); and

(4) it is stably maintained even in the absence of a drug owing to the uniform distribution (a typical plasmid is not stably maintained in a plasmodium parasites in the absence of a drug owing to non-uniform distribution and is finally lost from the Plasmodium parasites).

The base for the artificial chromosome of a Plasmodium may use, for example, a plasmid selected from pBR322, pUC18, pUC19, pUC118, pUC119, pBluescript (registered trade name), and the like using Escherichia coli as a host. The plasmid refers to an independent element used for the introduction of a nucleic acid into a cell.

The artificial chromosome of a Plasmodium contains a host Plasmodium-derived centromere region and may further contain a telomere region and exogenous genes such as a gene encoding a marker protein and a reporter gene. The exogenous genes include genes encoding traceable marker proteins, for example, genes encoding a green fluorescent protein (GFP), a yellow fluorescent protein (YFP), and a cyan fluorescent protein (CFP), and other reporter genes, for example, a chloramphenicol acetyltransferase (CAT) gene.

In the artificial chromosome of a Plasmodium, a sequence derived from a Plasmodium different from the host may be used for the sequence of an exogenous gene or its control region. The use of a sequence derived from a Plasmodium different from the host for the control region of an exogenous gene can suppress the incorporation of the introduced gene into the host chromosome; thus, the possibility is eliminated that the unexpected incorporation thereof into the host chromosome by homologous recombination occurs, enabling a precise experiment to be more reliably performed.

The “control region” of an exogenous gene refers to a region necessary for the expression of an exogenous gene in a Plasmodium or regulating the expression of the exogenous gene, and examples thereof include a promoter region and a transcription termination region.

In the artificial chromosome of a Plasmodium, the sequence of a centromere region, the sequence of a telomere region, the sequence of a control region, and the sequence of an exogenous gene are incorporated in a plasmid in an arrangement in which the artificial chromosome can function.

Examples of the host Plasmodium usable in the present invention include Plasmodium falciparum (P. falciparum), Plasmodium vivax (P. vivax), Plasmodium malariae (P. malariae), Plasmodium ovale (P. ovale), Simian malaria parasite (P. cynomolgi and P. knowlesi), and Rodent malaria parasite (P. berghei, P. chabaudi, and P. yoelii).

Examples of the Plasmodium different from the host, usable in the present invention include Plasmodium falciparum (P. falciparum), Plasmodium vivax (P. vivax), Plasmodium malariae (P. malariae), Plasmodium ovale (P. ovale), Simian malaria parasite (P. cynomolgi and P. knowlesi), and Rodent malaria parasite (P. berghei, P. chabaudi, and P. yoelii).

In one embodiment of the present invention, an artificial chromosome for Plasmodium berghei may be used, which uses Plasmodium berghei as a host and has the sequence (SEQ ID NO: 1) of a centromere region derived from chromosome 5.

In another embodiment, an artificial chromosome for Plasmodium falciparum may be used, which uses Plasmodium falciparum as a host and has any sequence selected from the sequences of centromere regions derived from chromosomes 1 to 9 and 11 to 14 or contains a sequence derived from Plasmodium berghei as the sequence of its control region. In the present specification, the sequences of centromere regions derived from chromosomes 5, 1, 2, 3, 4, 6, 7, 8, 9, 11, 12, 13, and 14 of Plasmodium falciparum are shown in SEQ ID NOS: 2 to 14 of the Sequence Listing, respectively.

In another embodiment, an artificial chromosome for Plasmodium vivax may be used, which uses Plasmodium vivax (P. vivax) as a host and has the sequence of a centromere region derived from a chromosome of Plasmodium vivax. In the present specification, the sequences deduced to be the sequences of centromere regions derived from chromosomes of Plasmodium vivax are shown in SEQ ID NOS: 16 to 19 of the Sequence Listing.

The artificial chromosome of a Plasmodium used in the present invention may be a circular artificial chromosome or a linear artificial chromosome. For the artificial chromosome of a Plasmodium berghei, the linear artificial chromosome has the advantage of having gene introduction efficiency 10 or more times higher than that of a typical circular plasmid, with no intention to limit the invention thereby; thus, the present invention can probably be more simply practiced.

### 3. Method for Screening for Drug-Resistant Gene

The present invention can use the artificial chromosome of a protozoa constructed according to item 2 above to screen for a drug-resistant gene by (1) preparing a recombinant protozoa into which an artificial chromosome containing candidate gene fragments is introduced, (2) inoculating the recombinant protozoa into a non-human mammal, followed by administering a drug, or culturing the recombinant protozoa in vitro and adding the drug to the culture system; and (3) recovering a drug-resistant recombinant protozoa from the non-human mammal or the culture system and identifying a candidate gene fragment contained in the protozoa as a drug-resistant gene. For example, when the artificial chromosome of a malaria parasite is used, the following steps can be performed to screen for a drug-resistant gene.

(1) Step of Preparing Recombinant Plasmodium into which Artificial Chromosome Containing Candidate Gene Fragment is Introduced

Candidate gene fragments are incorporated in an artificial chromosome of a Plasmodium using, for example, a DNA ligase such as T4 ligase. Ligation using the DNA ligase can be carried out according to a method well known to those skilled in the art. The artificial chromosome in which candidate gene fragments are incorporated is introduced into a wild-type host Plasmodium according to a method well known to those skilled in the art, for example, using an electroporation method. By the above step, a recombinant Plasmodium is prepared into which the artificial chromosome containing the candidate gene fragments is introduced.

Examples of the candidate gene fragments include DNA fragments derived from a Plasmodium exhibiting drug resistance, and sequences similar to drug-resistant genes of other protozoas.

The average length of the candidate gene fragments is 2.5 to 4.0 kb, preferably 4.0 to 10 kb, more preferably 10 to 50 kb. Use of large candidate gene fragments can improve the reliability of cloning of a drug-resistant gene.

In one embodiment of the present invention, the screening may be carried out by preparing a gene library for a Plasmodium exhibiting drug resistance and using the gene library as the artificial chromosome containing candidate gene fragments according to the present invention. In the embodiment, chromosomal DNA is first extracted from a Plasmodium exhibiting drug resistance, and the DNA is digested with a restriction enzyme to provide DNA fragments containing candidate gene fragments. The extraction and digestion of DNA can be carried out according to a method well known to those skilled in the art. Then, the resultant DNA fragments containing candidate gene fragments are each incorporated in an artificial chromosome of a Plasmodium using a DNA ligase to prepare a gene library. DNA ligases which may be used include, but not limited to, T4 ligase. Ligation using a DNA ligase can be carried out according to a method well known to those skilled in the art. The steps from the extraction of the chromosomal DNA to the preparation of the gene library can be performed in about 2 days.

The Plasmodium exhibiting drug resistance may be selected from the group consisting of Plasmodium falciparum (P. falciparum), Plasmodium vivax (P. vivax), Plasmodium malariae (P. malariae), Plasmodium ovale (P. ovale), Simian malaria parasite (P. cynomolgi and P. knowlesi), and Rodent malaria parasite (P. berghei, P. chabaudi, and P. yoelii).

(2) Step of Inoculating Recombinant Plasmodium into Non-Human Mammal, Followed by Administering Drug, or Step of Culturing it In Vitro Using Red Blood Cell to Infect Red Blood Cell Therewith, Followed by Adding Drug to Culture System

The recombinant Plasmodium into which an artificial chromosome containing candidate gene fragments is introduced is inoculated into a non-human mammal capable of being infected with the host Plasmodium immediately after the introduction to infect red blood cells therewith. Non-human mammals which may be used in the present invention include a rodent and a primate. For example, when an artificial chromosome of Plasmodium berghei is used, inoculation into mice or rats may be carried out, and when an artificial chromosome of Plasmodium falciparum is used, inoculation into monkeys can be carried out.

Alternatively, a recombinant Plasmodium into which an artificial chromosome containing candidate gene fragments is introduced is cultured in an in vitro culture system to infect red blood cells.

For Plasmodium falciparum, a continuous culture method has been established; thus, a recombinant Plasmodium can be cultured in vitro using the method (Trager, W. and Jensen. J. B. (1976) Human malaria protozoa in continuous culture. Science 193: 673-675).

After a lapse of a certain time from inoculation, a drug is administered or added to the non-human mammal into which a recombinant Plasmodium is inoculated or to the medium of the culture system. Examples of the drug usable here include therapeutic drugs for malaria such as chloroquine, quinine, pyrimethamine, mefloquine, primaquine, and artemisinin. For example, the administration of the drug may be started from 20 hours to 30 hours after inoculation when the artificial chromosome of Plasmodium berghei is used, and 48 hours after inoculation when Plasmodium falciparum is used. The dosage and frequency and period of administration of these drugs are properly adjusted for each drug by those skilled in the art.

In one embodiment of the present invention, when the gene library of a Plasmodium exhibiting drug resistance is used, a therapeutic drug for malaria to which the Plasmodium from which chromosomal DNA is extracted has exhibited resistance can be used as a drug.

(3) Step of Recovering Drug-Resistant Recombinant Plasmodium from Non-Human Mammal-Derived Infected Red Blood Cell and Infected Red Blood Cell in In Vitro Culture System and Identifying Candidate Gene Fragment Contained in Plasmodium as Drug-Resistant Gene

After inoculating the recombinant Plasmodium into a non-human mammal and administering a drug for a certain period, or after infecting red blood cells therewith in an in vitro culture system and adding the drug to the culture system for a certain period, the Plasmodium is recovered which survives in the animal body or the culture system. The drug administration eliminates a wild-type Plasmodium and the recombinant Plasmodium having no drug-resistant gene from the animal body or the culture system; thus, only the recombinant Plasmodium having acquired drug resistance is recovered in the step.

It takes about 5 to 20 days to perform the steps from the introduction of an artificial chromosome containing candidate gene fragments into a wild-type Plasmodium to the recovery of the recombinant Plasmodium having acquired drug resistance.

The DNA fragment containing the candidate gene fragment incorporated in the artificial chromosome of a Plasmodium, which is contained in the recovered recombinant Plasmodium, is excised using a restriction enzyme, sequenced, and identified as a drug-resistant gene. This step can be performed according to a method well known to those skilled in the art.

It takes about 2 days to perform the steps of recovering the artificial chromosome from the recovered recombinant Plasmodium and identifying the base sequence of the candidate gene fragment.

### 4. Method for Preparing Recombinant Plasmodium

The use of the present invention can prepare a recombinant Plasmodium into which an artificial chromosome is introduced more rapidly and more simply than before, using the artificial chromosome of a Plasmodium constructed according to the above item 2. 2.

(1) Step of Preparing Schizont-Stage Plasmodium Parasite at Stage Immediately Before Entry into Red Blood Cell

(i) Purification of Schizont-Stage Plasmodium Parasite

The culture of a Plasmodium is centrifuged to remove the supernatant to prepare such a cell suspension that contains the Plasmodium at a high concentration. The cell suspension is subjected to density gradient centrifugation to separate and purify the Plasmodium for each particular stage during the life cycle, and a schizont-stage Plasmodium parasite located in the intermediate layer is recovered. In comparison between a ring-stage Plasmodium parasite and a schizont-stage Plasmodium parasite, it is known that the schizont-stage Plasmodium parasite generally has a lower specific gravity. The schizont-stage Plasmodium parasite can be selected using the division and shape of the nucleus as an index by a fluorescence microscope observation. The schizont-stage Plasmodium parasite can also be selected using the division and shape of the nucleus as an index by a microscope observation after Giemsa staining.

Media for forming the density gradient include, for example, sucrose, glycerol and Nycodenz (registered trade name, from Axis-Shield PoC AS); both ionic and non-ionic ones may also be used. Media commercially available for density gradient formation may be suitably used such as Ficoll (registered trade name), Ficoll-Paque (registered trade name), and Percoll (registered trade name) (all are from GE Healthcare Japan). Preferably, Percoll or Nycodenz is used.

When Percoll is used as a medium for forming the density gradient, sorbitol is also added to prepare a Percoll/sorbitol solution. This is diluted with an incomplete medium to prepare a dilution series, which are then layered in a tube to prepare a density gradient.

The cell suspension containing plasmodium parasite is layered on the medium forming a density gradient, followed by centrifugation.

(ii) Sorbitol Treatment

In one embodiment of the present invention, the Plasmodium purified in item (i) above is cultured together with red blood cells in a complete medium at 37° C. in the presence of a mixed gas (N₂: 90%, O₂: 5%, CO₂: 5%). After 4 hours of culture, the culture is centrifuged to recover red blood cells. The recovered red blood cells receive the addition of a 10-fold volume (v/v) of a sorbitol solution and are allowed to stand at 37° C. The resultant was again centrifuged to recover red blood cells, which then receive the addition of a 10-fold volume (v/v) of an incomplete medium before suspension. Thereafter, the resultant was again centrifuged to recover red blood cells, to which the complete medium is then added, followed by culture at 37° C. During this process, the cell cycle is synchronized to the ring-stage Plasmodium parasite immediately to 4 hours after the infection of red blood cells. In addition, culture at 37° C. can be continued for about 96 hours to provide a large amount of the schizont-stage Plasmodium parasite.

The above steps (i) and (ii) can be repeated to perform the highly synchronous culture of the Plasmodium.

(2) Step of Directly Introducing Artificial Chromosome into Schizont-Stage Plasmodium Parasite at Stage Immediately Before Entry into Red Blood Cell by Electroporation

The above step (i) is again performed for the Cell suspension containing the highly synchronously cultured Plasmodium, obtained in item (1) above to purify the schizont-stage plasmodium parasite. The purified plasmodium parasite is suspended in a 50-fold (v/v) or more of the complete medium and cultured at 37° C. in the presence of a mixed gas (N₂: 90%, O₂: 5%, CO₂: 5%).

Nuclear staining is performed with Hoechst33342 at one-hour intervals after the start of culture, and the nuclear division, shape, and number, the parasitophorous vacuole membrane, and the red blood cell membrane are observed. The appearance of many mature schizont-stage plasmodium parasites is timed each of which has a nuclear number of 16 or 32 and also has either of the parasitophorous vacuole membrane and the red blood cell membrane collapsed.

Immediately after determining the timing at which many mature schizont-stage plasmodium parasites appear by observation, the plasmodium parasites are recovered, followed by introducing DNA by electroporation. The electroporation can be carried out according to a method well known to those skilled in the art; for example, a reagent and a device for electroporation which are commercially available, such as T-cell nucleofector and Nucleofector II (from Lonza) can be used.

After the end of electroporation, culture is performed for proliferation at 37° C. in a complete medium containing red blood cells in the presence of a mixed gas (N₂: 90%, O₂: 5%, CO₂: 5%). Subsequently at 24-hour intervals, some infected red blood cells are collected and subjected to Giemsa staining to monitor the percentage of parasitism of plasmodium parasites.

(3) Feature and Advantage

The above steps can introduce an exogenous gene into a Plasmodium more efficiently than conventional methods and can prepare a recombinant Plasmodium in a shorter period of time.

A mechanism by which a Plasmodium appears from the red blood cell membrane probably does not greatly vary among all Plasmodia; thus, the method of the present invention, that is, the method for preparing a mature schizont-stage plasmodium parasite and directly introducing an exogenous gene thereinto by electroporation can be applied to Plasmodium falciparum (P. falciparum), Plasmodium vivax (P. vivax), Plasmodium malariae (P. malariae), Plasmodium ovale (P. ovale), Simian malaria parasite (P. cynomolgi and P. knowlesi), and the like. The method of the present invention is preferably applied to Plasmodium falciparum.

The genus Toxoplasma, the genus Theileria, the genus Babesia, and the genus Plasmodium belonging to the phylum Apicomplexa are present in positions evolutionarily close to each other, have similar life cycles in terms of having a life cycle consisting of sexual reproduction and asexual reproduction and forming a cyst, and also have similar properties in the cell structure in terms of having a common cell organ called an apical complex. Thus, the method of the present invention can probably be widely applied to the above protozoas, that is, protozoas of the genus Toxoplasma, protozoas of the genus Theileria, protozoas of the genus Babesia, and protozoas of the genus Plasmodium belonging to the phylum Apicomplexa as well as the genus Plasmodium.

Use of schizont-stage Plasmodium falciparum has previously been believed to improve the gene introduction efficiency because the plasmodium parasites at the stage can infect a red blood cell immediately after gene introduction and many plasmodium parasites are present in the infected red blood cell. However, because electroporation causes the death thereof, they could not actually be used for the gene introduction.

The present inventor has focused attention on the known finding that among schizont-stage Plasmodium falciparum, a plasmodium parasite at a more particular stage (defined herein as the mature schizont-stage or the schizont-stage at a stage immediately before entry into red blood cells) has the number of membranes in the periphery of the plasmodium parasite decreased by one, and performed gene introduction using a schizont-stage plasmodium parasite at the particular stage. As a result, it has been discovered that (1) the use of the schizont-stage plasmodium parasite at the particular stage improves the efficiency of gene introduction due to the decrease in the number of membranes in the periphery of the plasmodium parasite by one and (2) the schizont-stage plasmodium parasite at the particular stage has the feature that it does not die due to electroporation. A new method has also been developed for preparing a schizont-stage Plasmodium falciparum at a stage immediately before entry into red blood cells, having such features.

The use of the method of the present invention can prepare the schizont-stage plasmodium parasite at a stage immediately before entry into red blood cells in large amounts. A schizont-stage plasmodium parasite is present in large numbers in one red blood cell compared to plasmodium parasites at other stages; thus, from such a point, the method of the present invention can be said to enable the preparation of a Plasmodium usable for gene introduction in large amounts.

The method of the present invention enables a gene to be introduced into a Plasmodium falciparum 1,000 or more times more efficiently than conventional methods. Here, the improvement of the gene introduction efficiency is calculated as follows.

A Plasmodium falciparum multiplies on the order of 10 times in one cell cycle (48 hours, i.e. 2 days); thus, the improvement can be determined by the equation:

Ratio of Improvement of Gene Introduction=10^{(y-x)/2}

(where y is the number of days required for exceeding a certain percentage of parasitism for a conventional method (an indirect introduction method) and x is the number of days required for exceeding a certain percentage of parasitism for the method of the present invention (a direct introduction method)).

The use of the method of the present invention can introduce an exogenous gene into a Plasmodium at a high efficiency; thus, it can decrease the amount of DNA used for the introduction compared to conventional methods.

The present invention will be described below in further detail with reference to Examples. However, these Examples are not intended to limit the present invention.

### Example

### 1. Screening for Drug-Resistant Gene Using Completely Digested Plasmodium Berghei Chromosomal DNA Containing Known Drug-Resistant Gene

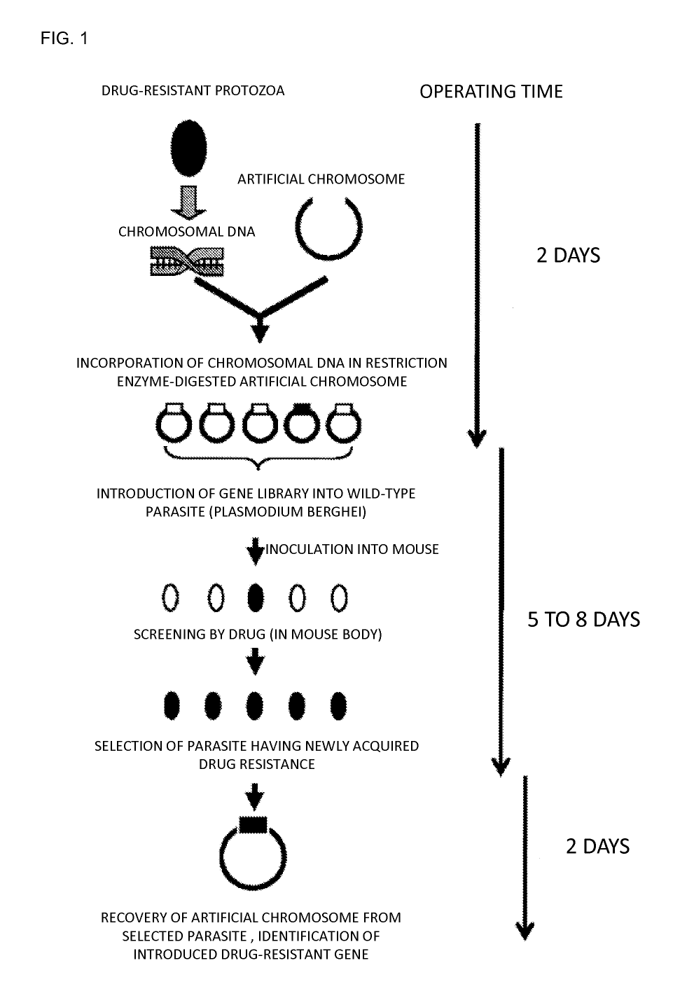

The following experiment was carried out according to the schematic of a method for screening for a drug-resistant gene of a Plasmodium shown in FIG. 1.

The drug-resistant plasmodium parasite used was a Plasmodium berghei in which a drug-resistant gene was artificially incorporated at a particular position on the plasmodium parasite chromosome using homologous recombination. The drug-resistant gene used was a human-derived dihydrofolate reductase; the gene imparts pyrimethamine resistance to the Plasmodium.

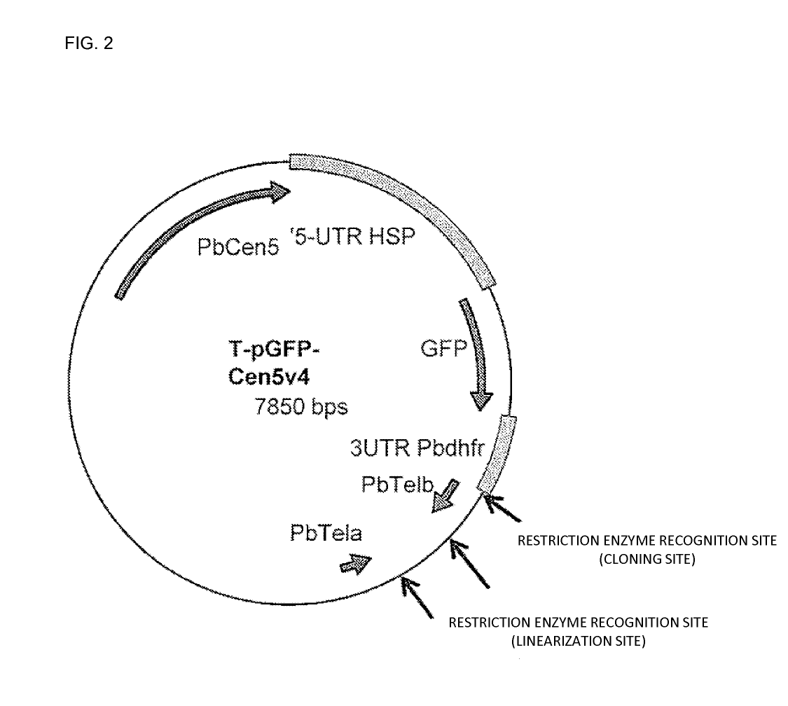

The artificial chromosome of a Plasmodium berghei (SEQ ID NO: 15) shown in FIG. 2 was used for the experiment. In the artificial chromosome of a Plasmodium berghei are incorporated a centromere sequence derived from chromosome 5 of Plasmodium berghei (PbCen5), 5′-UTR HSP of Plasmodium berghei (5′ UTR of heat shock protein), Aequorea-derived GFP, 3′-UTR Pbdhfr of Plasmodium berghei (3′UTR of dihydrofolate reductase), and telomere sequences of Plasmodium berghei (PbTela and PbTelb).

(1) Preparation of Gene Library

Plasmodium parasite chromosomal DNA was extracted and purified from the drug-resistant plasmodium parasite and completely digested with a restriction enzyme (HindIII). The complete digestion of the plasmodium parasite chromosome is known to provide a candidate gene fragment of 2,966 bp containing a drug-resistant gene (dihydrofolate reductase); thus, the digested product was subjected to agarose gel electrophoresis to separate DNA fragments having sizes ranging from about 2.5 kb to about 4.0 kb, which were then recovered from the gel.

The artificial chromosome was completely digested with the same restriction enzyme as that used for the plasmodium parasite chromosomal DNA, and treated with alkaline phosphatase to prevent self-cyclization during ligation reaction for terminal dephosphorylation.

Subsequently, the DNA fragments of about 2.5 to about 4.0 kb containing the candidate gene fragments were mixed with the artificial chromosome digested with the restriction enzyme, which was then subjected to ligation reaction. Thereafter, protein in the reaction solution was removed by phenol/chloroform/isoamyl alcohol treatment, followed by ethanol precipitation for the recovery of an artificial chromosome in which the DNA fragments were incorporated by the ligation reaction. Through the above operations, a gene library using the drug-resistant plasmodium parasite-derived chromosomal DNA and the artificial chromosome was constructed.

Then, the recovered artificial chromosome was digested with a restriction enzyme, PmeI, for linearization and the gene library was introduced into a plasmodium parasite by electroporation. A drug-sensitive, i.e. wild-type plasmodium parasite was used for the gene introduction.

(2) Screening for Drug-Resistant Gene

The plasmodium parasite having the gene library introduced was inoculated into mice, to which a drinking water containing pyrimethamine (final concentration: 7 μg/ml) was given from 20 to 30 hours after inoculation to screen for a drug-resistant plasmodium parasite. During the period of drug administration, blood smears were prepared at 24-hour intervals and subjected to Giemsa staining to detect the plasmodium parasite.

The appearance of a recombinant plasmodium parasite having newly acquired drug resistance was confirmed at a stage at which 5 to 6 days lapsed from the start of drug administration. At a stage at which the percentage of parasitism of plasmodium parasites increased to on the order of 5 to 100, the selected drug-resistant plasmodium parasite was purified, and chromosomal DNA was extracted and purified to recover the introduced artificial chromosome. The “percentage of parasitism of plasmodium parasites” is expressed as a percentage by calculating the proportion of red blood cells infected by Plasmodium in all red blood cells.

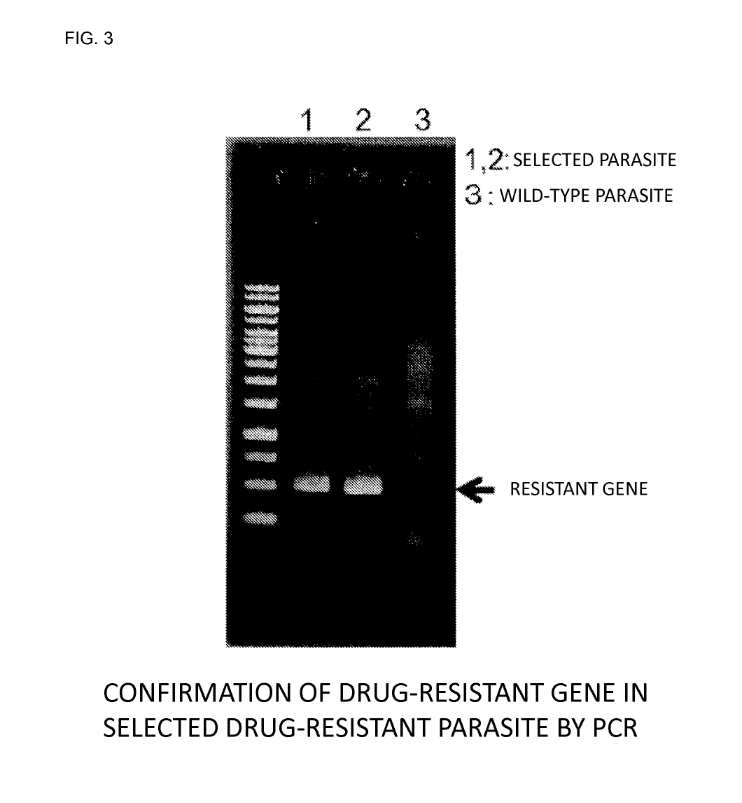

It was confirmed by PCR that the artificial chromosome was introduced into the selected recombinant plasmodium parasite and the human-derived dihydrofolate reductase (drug-resistant gene) was introduced therein (FIG. 3).

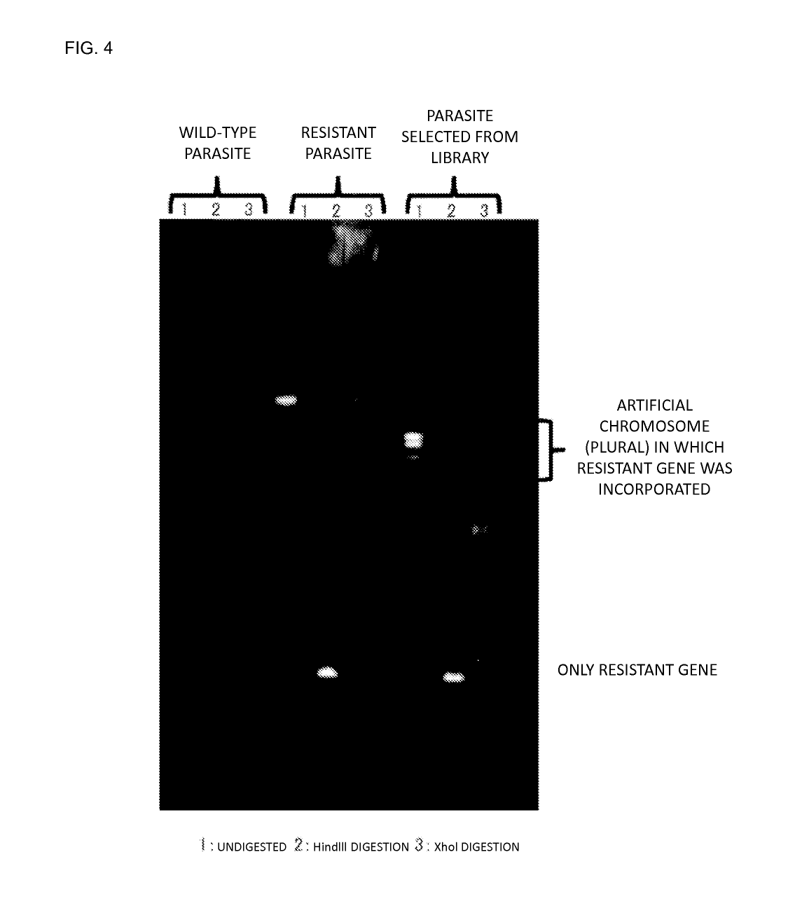

In addition, by Southern hybridization using the dihydrofolate reductase gene as a probe, it was confirmed that the drug-resistant gene was incorporated in the artificial chromosome and DNA fragments having a plurality types of lengths containing the drug-resistant gene were incorporated in the artificial chromosome (FIG. 4).

The above experiment demonstrated that a gene library using drug-resistant plasmodium parasite-derived chromosomal DNA and the Plasmodium artificial chromosome could be prepared to rapidly identify the resistant gene.

### 2. Screening for Drug-Resistant Gene Using Partially Digested Plasmodium berghei Chromosomal DNA Containing Known Drug-Resistant Gene

(1) Preparation of Gene Library

Plasmodium parasite chromosomal DNA was extracted and purified from a drug-resistant plasmodium parasite and partially digested with a restriction enzyme (HindIII). The digested product was subjected to electrophoresis using a low melting point agarose gel to separate DNA fragments having sizes of about 10 kb to about 50 kb, which were then treated with β-agarase, followed by recovering the DNA fragments. It was confirmed by PCR that the intended drug-resistant gene was contained in the recovered DNA fragments.

For subsequent preparation of an artificial chromosome, construction of a gene library, and introduction of the gene library into a wild-type plasmodium parasite, operations were carried out according to the materials and methods shown in item 1. above.

(2) Screening for Drug-Resistant Gene

The plasmodium parasite having the gene library introduced was inoculated into mice, to which a drinking water containing pyrimethamine (final concentration: 7 μg/ml) was given from 20 to 30 hours after to screen for a drug-resistant plasmodium parasite. During the period of drug administration, blood smears were prepared at 24-hour intervals and subjected to Giemsa staining to detect plasmodium parasites.

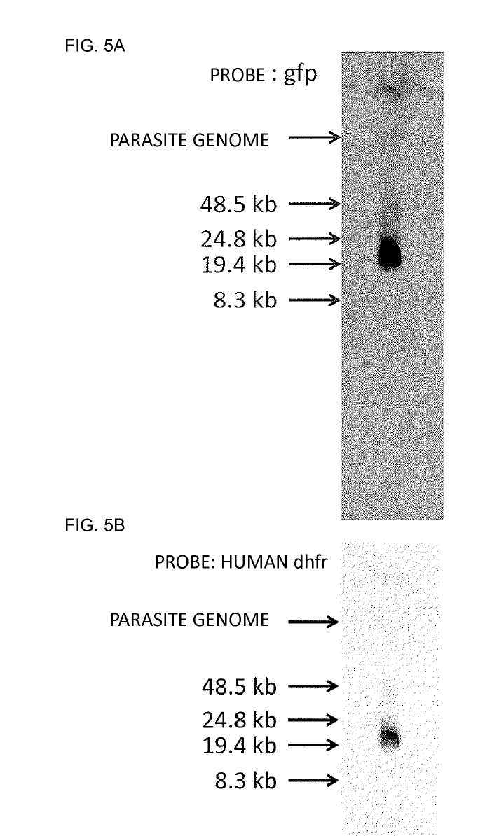

The appearance of a recombinant plasmodium parasite newly acquired drug resistance was confirmed at a stage at which 5 to 6 days lapsed from the start of drug administration. At a stage at which the percentage of parasitism of plasmodium parasites increased to on the order of 5 to 100, the selected drug-resistant plasmodium parasite was purified and embedded in a low melting point agarose gel to prepare a plasmodium parasite agarose block. This was subjected to CHEF (Clamped Homogeneous Electric Fields) electrophoresis, and thereafter, by Southern hybridization using dihydrofolate reductase gene and GFP gene as probes, it was confirmed that the artificial chromosome was present in the selected plasmodium parasite and the drug-resistant gene was incorporated in the artificial chromosome (FIG. 5).

Because GFP gene is incorporated in the artificial chromosome used, it can be used as a probe to detect only the artificial chromosome. In addition, from the results of the Southern hybridization, it was confirmed that DNA fragments having sizes of at least on the order of 10 kb to 15 kb and containing the drug-resistant gene were incorporated in the artificial chromosome.

### 3. Preparation of Gene Library Using Partially Digested Drug-Resistant Plasmodium falciparum-Derived Chromosomal DNA

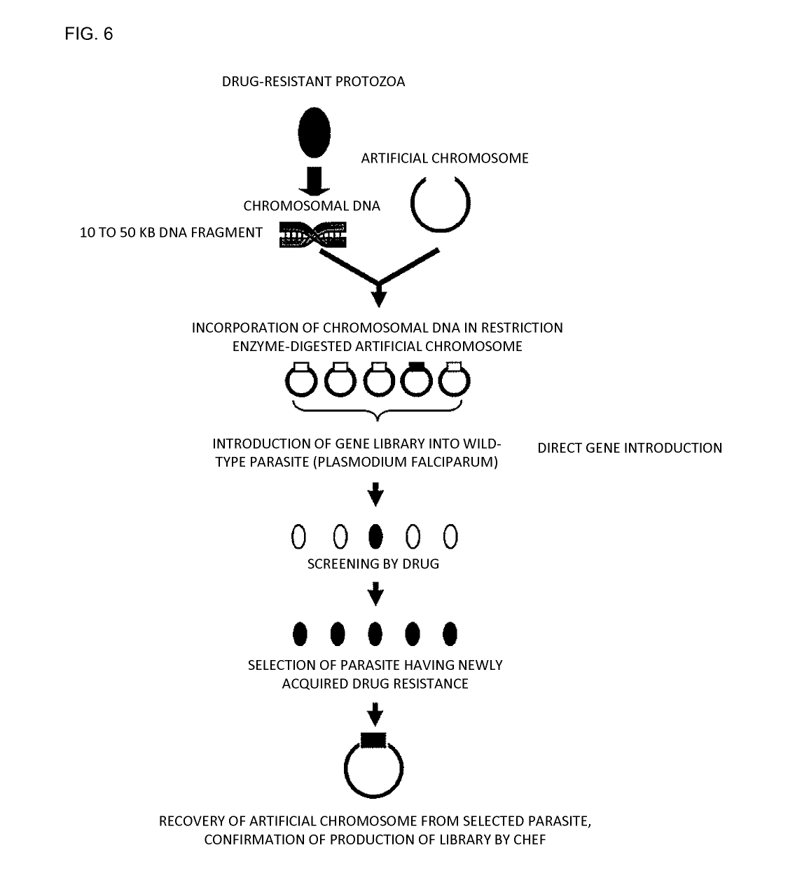

The following experiment was carried out for the purpose of the preparation of a gene library included in the operation process of the method for screening for a drug-resistant gene of Plasmodium falciparum, shown in FIG. 6.

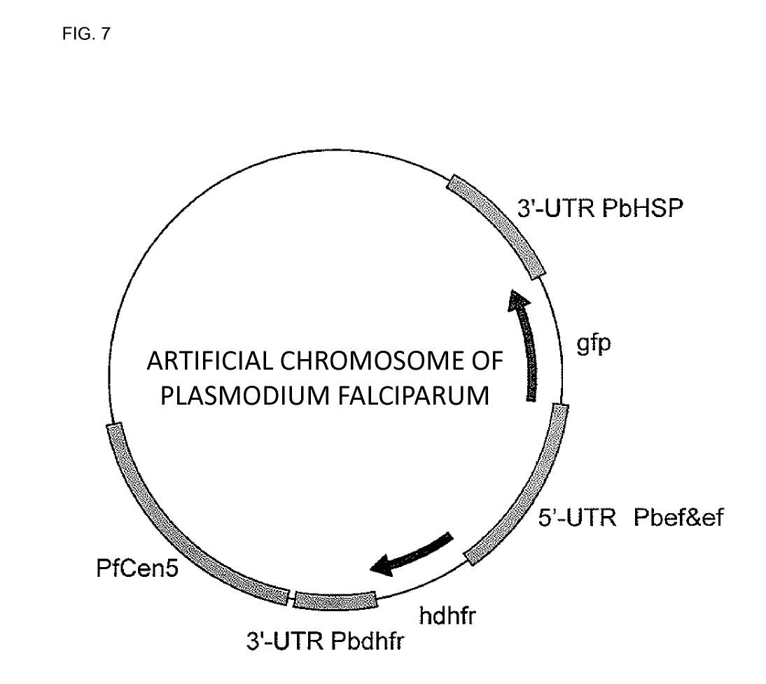

The artificial chromosome of Plasmodium falciparum (SEQ ID NO: 20) shown in FIG. 7 was used for the experiment. In the artificial chromosome of Plasmodium falciparum are incorporated a centromere sequence derived from chromosome 5 of Plasmodium falciparum (PfCen5), 3′-UTR PbHSP (3′-UTR of Plasmodium berghei-derived heat shock protein), Aequorea-derived GFP, 3′-UTR Pbdhfr (3′-UTR of Plasmodium berghei-derived dihydrofolate reductase), human-derived dihydrofolate reductase (hdhfr), and 5′-UTR Pbef&ef (5′-UTR of Plasmodium berghei-derived elongation factor sequence). Incidentally, hdhfr imparts pyrimethamine resistance to a Plasmodium.

(1) Preparation of Gene Library

Plasmodium parasite chromosomal DNA was extracted and purified from a drug-resistant Plasmodium parasite (a chloroquine-resistant plasmodium parasite) and partially digested with a restriction enzyme (BamHI). The digested product was subjected to agarose gel electrophoresis to separate DNA fragments having sizes ranging from about 10 kb to about 50 kb before β-agarase treatment, followed by recovering the DNA fragments from the gel.

The artificial chromosome was completely digested with the same restriction enzyme as that used for the plasmodium parasite chromosomal DNA, and treated with alkaline phosphatase to prevent self-cyclization during ligation reaction for terminal dephosphorylation.

Subsequently, the DNA fragments of about 10 to about 50 kb containing the candidate gene fragments were each mixed with the artificial chromosome digested with the restriction enzyme, which was then subjected to ligation reaction. Thereafter, protein in the reaction solution was removed by phenol/chloroform/isoamyl alcohol treatment, followed by ethanol precipitation for the recovery of an artificial chromosome in which the DNA fragments were incorporated by the ligation reaction. Through the above operations, a gene library using the drug-resistant plasmodium parasite (a chloroquine-resistant plasmodium parasite)-derived chromosomal DNA and the artificial chromosome was constructed.

(2) Introduction of Gene Library into Plasmodium Parasite (Direct Introduction Method)

Then, by a direct introduction method, the gene library was introduced into a drug-sensitive plasmodium parasite, i.e. a wild-type Plasmodium falciparum. The direct introduction method was carried out as follows.

(i) Step 1: Schizont Purification

Reagent

1: Incomplete medium: RPMI1640 (from Invitrogen), 25 mM HEPS, 0.005% hypoxanthine 2: Complete medium: RPMI1640, 25 mM HEPS, 0.005% hypoxanthine, 0.225% sodium hydrogen carbonate, 10 mg/ml gentamicin, 0.25% AlbumaxI (registered trade name) (from Invitrogen)

3: 90% Percoll/sorbitol solution: 90 ml Percoll solution, 10 ml 0×RPMI solution, 6 g sorbitol (this was used as a stock solution and diluted with the incomplete medium to prepare 70% and 40% Percoll/sorbitol solutions).

Procedure

A culture solution (2% hematocrit (Ht), 40 ml) infected by Plasmodium falciparum (percentage of parasitism: 5 to 100) was centrifuged at 3,000 rpm for 5 minutes, and the supernatant was removed until the remaining reaches on the order of 1.5 ml.

3 ml of 70% Percoll/sorbitol was placed in a 15-ml tube, and 3 ml of 40% Percoll/sorbitol was layered thereon. Further thereon, 1.5 ml of the Plasmodium culture prepared above was layered.

The resultant was centrifuged at 3,500 rpm for 25 minutes, and a schizont-stage plasmodium parasite located in the intermediate layer was finally recovered.

(ii) Step 2: Sorbitol Treatment

Reagent

1: 5% sorbitol

Procedure

To the schizont-stage plasmodium parasite purified in the step 1 was added a 20-fold volume (v/v) of red blood cells (Ht, 500), which was then diluted with the complete medium so that it finally has an Ht concentration of 5%. Thereafter, the resultant was cultured at 37° C. for 4 hours in the presence of a mixed gas (N2: 90%, O2: 5%, CO2: 5%).

After 4 hours, the resultant was centrifuged at 3,000 rpm for 5 minutes. To the recovered red blood cells was added a 10-fold volume (v/v) of a 5% sorbitol solution, which was then allowed to stand at 37° C. for 8 minutes.

The resultant was centrifuged at 2,500 rpm for 2 minutes, and a 10-fold volume (v/v) of the incomplete medium was added to the recovered red blood cells before suspension. Thereafter, the suspension was again centrifuged at 3,000 rpm for 5 minutes.

After centrifugation, the complete medium was added to the recovered red blood cells so that the resultant has an Ht concentration of 2%. During this process, the cell cycle is synchronized to the ring-stage plasmodium parasite immediately to 4 hours after the infection of red blood cells.

Subsequently, culture is carried out at 37° C. for about 96 hours. This results in that a large amount of plasmodium parasites become schizont-stage plasmodium parasites.

(iii) Step 3: Direct Gene Introduction

Procedure

After the end of culture in the step 2, the steps 1 and 2 were again repeated. The repeated operation established a population of highly synchronously cultured schizont-stage Plasmodium falciparum.

Then, only the step 1 was again carried out to purify the schizont-stage plasmodium parasite, and the resultant was suspended in a 50-fold or more volume (v/v) of the complete medium and cultured at 37° C. in the presence of a mixed gas (N₂: 90%, O₂:5%, CO₂: 5%).

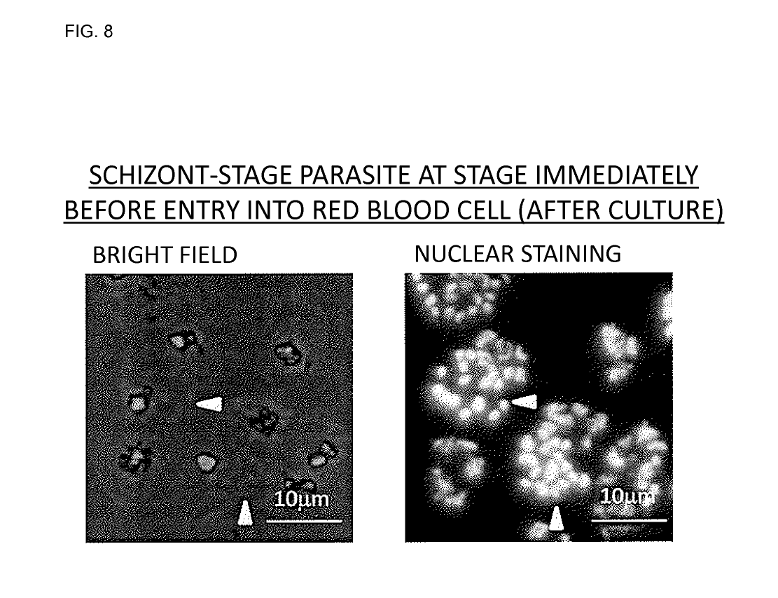

Nuclear staining with Hoechst33342 was performed at one-hour intervals from the start of culture, and the division and shape of nuclei, the parasitophorous vacuole membrane, and the red blood cell membrane were observed (FIG. 8). Through such an observation, the appearance of many mature schizont-stage plasmodium parasites was timed each of which has the parasitophorous vacuole membrane or the red blood cell membrane collapsed.

Immediately after the determination of timing by the observation, 1×10⁸ mature schizont-stage plasmodium parasites were recovered, and mixed with 100 ml of T-cell nucleofector (from Lonza) containing DNA (5 mg to 50 mg).

Immediately after mixing, Nucleofector II (U-33 program) was used to perform electroporation. After the end thereof, 100 ml of the complete medium was added thereto, which was then transferred to 5 ml of the complete medium having an Ht concentration of 20, followed by starting culture at 37° C. in the presence of a mixed gas (N2: 90%, O2: 5%, CO2: 5%). Subsequently at 24-hour intervals, some infected red blood cells are collected and subjected to Giemsa staining to calculate the percentage of parasitism of plasmodium parasites.

(3) Screening for Plasmodium Parasite into which Gene Library was Introduced

The wild-type Plasmodium falciparum into which the gene library was introduced by the above direct introduction method were cultured using a pyrimethamine-containing medium. All the plasmodium parasites into which the artificial chromosome was introduced become pyrimethamine-resistant due to the presence of hdhfr gene; thus, the culture thereof in the presence of pyrimethamine enabled all the plasmodium parasites having the gene library using the artificial chromosome introduced to be selected. Plasmodium parasite into which the gene library was not introduced in the presence of pyrimethamine died out because of not acquiring drug resistance.

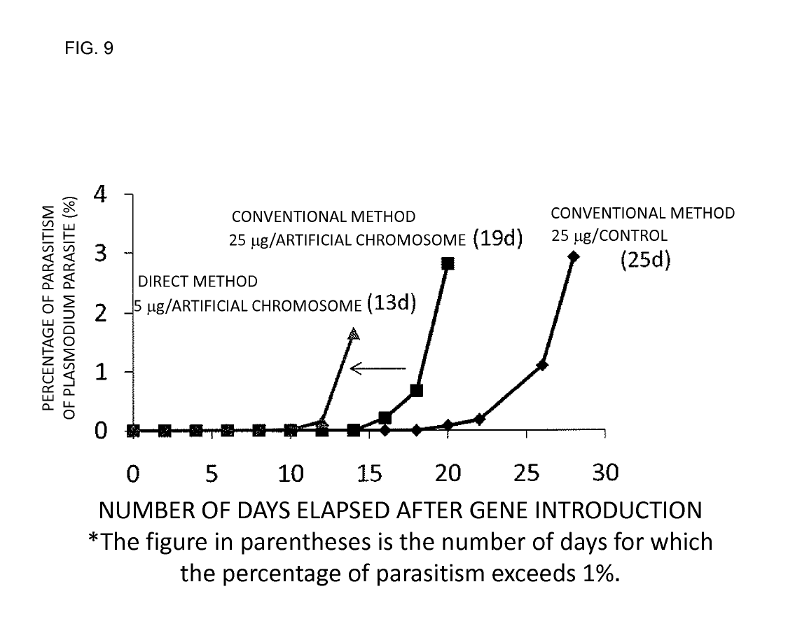

As a control for comparing the efficiency of gene introduction, the change of the percentage of parasitism of plasmodium parasites was similarly monitored to measure the number of days required for that the percentage of parasitism of plasmodium parasites exceeds 1% in cases where 25 μg of a control plasmid containing no centromere sequence (that is, a plasmid which is not an artificial chromosome) was indirectly introduced and where 25 μg of the artificial chromosome was indirectly introduced. The number of such days was 25 for the indirect introduction of the control plasmid, 19 for the indirect introduction of the artificial chromosome, and 13 for the use of the method of the present invention (FIG. 9). Thus, the use of the method of the present invention was shown to improve the gene introduction efficiency 10⁶ (10^{(25-13)/2}) times compared to no use of the artificial chromosome and 10³ (10^{(19-13)/2}) times compared to the indirect introduction of the artificial chromosome.

In addition, the amount of the artificial chromosome used was 25 μg for the indirect introduction thereof, while being 5 μg for the direct introduction thereof; thus, the direct introduction according to the present invention can be said to have a 10³-fold or more introduction efficiency compared to the conventional indirect introduction method.

(4) Confirmation of Library Construction by CHEF

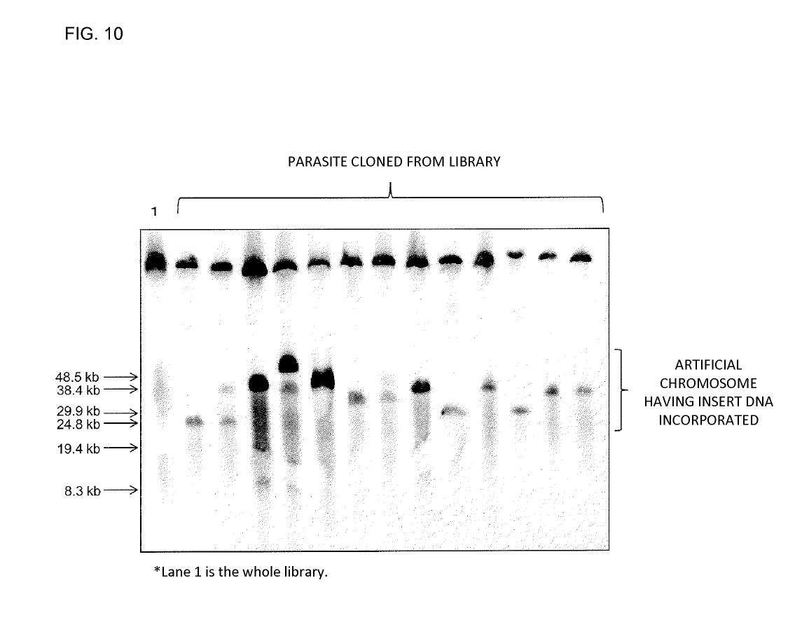

Individual plasmodium parasites were cloned from the population of the plasmodium parasites having the gene library selected in item (3) above introduced, and the size of the artificial chromosome incorporated in each plasmodium parasite clone was confirmed by CHEF (contour-clamped homogeneous electric field). The artificial chromosome was detected by Southern hybridization using hdhfr gene as a probe (FIG. 10). As a result, since the size of the incorporated artificial chromosome was different for each of the clones, it was confirmed that the artificial chromosome having a different insert DNA was introduced into the plasmodium parasite, that is, a library of plasmodium parasite DNA was produced. The detection of a plurality of signals on one sample (lane) indicates the possibility that a plasmodium parasite has 2 types of artificial chromosomes or 2 types of clones are mixed with each other.

### 4. Screening for Drug-Resistant Gene (Chloroquine-Resistant Gene)

The wild-type Plasmodium falciparum into which the gene library derived from a drug-resistant Plasmodium falciparum (a chloroquine-resistant plasmodium parasite) was introduced, described in term 3 above is cultured in vitro in the presence of red blood cells. The culture can be carried out by adding chloroquine to the medium to select a drug-resistant Plasmodium falciparum having an artificial chromosome having a chloroquine-resistant gene incorporated.

The artificial chromosome is recovered from the selected plasmodium parasite and sequenced to identify the chloroquine-resistant gene.

A gene library using a chromosomal DNA derived from a Plasmodium falciparum resistant to another therapeutic drug for malaria (quinine, pyrimethamine, mefloquine, primaquine, artemisinin, or the like) can be prepared according to the method described in term 3 above and subjected to the same screening as that described above using the appropriate drug to identify a drug-resistant gene.

### INDUSTRIAL APPLICABILITY

According to the present invention, a drug-resistant gene of a Plasmodium can be rapidly and precisely identified to elucidate the resistance mechanism to contribute to the development research of a new drug effective even for the resistant plasmodium parasite. According to the present invention, a drug-resistant gene of a Plasmodium can also be rapidly and precisely identified in a field to monitor the distribution of a resistant plasmodium parasite to perform effective medication as well as to delay the further appearance of the resistant plasmodium parasite. In addition, the method of the present invention enables a recombinant Plasmodium to be prepared more efficiently in a shorter period of time and thus can further facilitate the identification of the drug-resistant gene of a Plasmodium.

All publications, patents, and patent applications cited in this application are intended to be incorporated herein by reference in their entirety.

1. A method for screening for a drug-resistant gene, comprising the steps of:
(a) digesting chromosomal DNA extracted from a protozoa exhibiting drug resistance with a restriction enzyme to prepare a plurality of candidate gene fragments, followed by incorporating the candidate gene fragments into an artificial chromosome;
(b) preparing a recombinant protozoa into which the artificial chromosome containing the candidate gene fragments is introduced;
(c) inoculating the recombinant protozoa into a non-human mammal, followed by administering a drug, or culturing the recombinant protozoa in vitro and adding the drug to the culture system; and
(d) recovering a drug-resistant recombinant protozoa from the non-human mammal or the culture system to identify a candidate gene fragment contained in the protozoa as a drug-resistant gene,
wherein the artificial chromosome is an artificial chromosome of a protozoa containing a protozoa-derived centromere region.

2. The method for screening for a drug-resistant gene according to claim 1, comprising the steps of:
(a) digesting chromosomal DNA extracted from a Plasmodium parasite exhibiting drug resistance with a restriction enzyme to prepare a plurality of candidate gene fragments, followed by incorporating the candidate gene fragments into an artificial chromosome;
(b) preparing a recombinant Plasmodium parasite into which the artificial chromosome containing the candidate gene fragments is introduced;
(c) inoculating the recombinant Plasmodium parasite into a non-human mammal, followed by administering a drug; and
(d) recovering a drug-resistant recombinant Plasmodium parasite from the non-human mammal and identifying a candidate gene fragment contained in the Plasmodium parasite as a drug-resistant gene,
wherein the artificial chromosome is an artificial chromosome of a Plasmodium containing a Plasmodium parasite-derived centromere region.

3. The method for screening for a drug-resistant gene according to claim 1, comprising the steps of:
(a) digesting chromosomal DNA extracted from a Plasmodium parasite exhibiting drug resistance with a restriction enzyme to prepare a plurality of candidate gene fragments, followed by incorporating the candidate gene fragments into an artificial chromosome;
(b) preparing a recombinant Plasmodium parasite into which the artificial chromosome containing the candidate gene fragments is introduced;
(c) culturing the recombinant Plasmodium parasite in vitro using red blood cells to infect the red blood cells therewith, followed by adding a drug to the culture system; and
(d) recovering a drug-resistant recombinant Plasmodium parasite from the culture system and identifying a candidate gene fragment contained in the Plasmodium parasite as a drug-resistant gene,
wherein the artificial chromosome is an artificial chromosome of a Plasmodium containing a Plasmodium parasite-derived centromere region.

4. The method according to claim 1, wherein the non-human mammal is a rodent or a primate.

5. The method according to claim 2, wherein the artificial chromosome of a Plasmodium parasite is one containing a centromere region derived from Plasmodium berghei.

6. The method according to claim 3, wherein the artificial chromosome of a Plasmodium parasite is a circular artificial chromosome.

7. The method according to claim 2, wherein the artificial chromosome of a Plasmodium parasite is a linear artificial chromosome.

8. The method according to claim 2, wherein the candidate gene fragments are gene fragments derived from a drug-resistant Plasmodium parasite.

9. (canceled)

10. The method according to claim 1, wherein the candidate gene fragments have an average length of 4.0 to 10 kb.

11. The method according to claim 1, wherein the candidate gene fragments have an average length of 10 to 50 kb.

12. The method according to claim 1, wherein the drug is a therapeutic drug for malaria.

13. The method according to claim 12, wherein the therapeutic drug for malaria is a drug selected from the group consisting of chloroquine, quinine, pyrimethamine, mefloquine, primaquine, and artemisinin.

14. A method for preparing a recombinant Plasmodium parasite, comprising the steps of:
(a) preparing a schizont-stage Plasmodium parasite at a stage immediately before entry into red blood cells; and
(b) directly introducing an artificial chromosome into the schizont-stage Plasmodium parasite at the stage immediately before entry into red blood cells by electroporation,
wherein the artificial chromosome is an artificial chromosome of a Plasmodium containing a Plasmodium parasite-derived centromere region, and the Plasmodium parasite is selected from the group consisting of Plasmodium falciparum, Plasmodium vivax, Plasmodium marariae, Plasmodium ovale, and Simian malaria parasite (P. cynomolgi and P. knowlesi).

15. (canceled)

16. The method according to claim 3, wherein the recombinant Plasmodium parasite is prepared using the method comprising the steps of:
(a) preparing a schizont-stage Plasmodium parasite at a stage immediately before entry into red blood cells; and
(b) directly introducing an artificial chromosome into the schizont-stage Plasmodium parasite at the stage immediately before entry into red blood cells by electroporation,
wherein the artificial chromosome is an artificial chromosome of a Plasmodium containing a Plasmodium parasite-derived centromere region, and the Plasmodium parasite is selected from the group consisting of Plasmodium falciparum, Plasmodium vivax, Plasmodium marariae, Plasmodium ovale, and Simian malaria parasite (P. cynomolgi and P. knowlesi).

17. (canceled)

18. The method according to claim 3, wherein the artificial chromosome of a Plasmodium parasite is one containing a centromere region derived from Plasmodium falciparum or Plasmodium vivax.

19. The method according to claim 3, wherein the candidate gene fragments are gene fragments derived from a drug-resistant Plasmodium parasite.
